(19) United States
(12) Patent Application Publication
(10) Pub. No.: US 2013/0252983 A1
(43) Pub. Date: Sep. 26, 2013

(54) ACTIVATING PHOSPHORYLATION SITE ON GLUTAMINASE C

(71) Applicants: Kristin Wilson Cerione, Ithaca, NY (US); Richard A. Cerione, Ithaca, NY (US)

(72) Inventors: Kristin Wilson Cerione, Ithaca, NY (US); Richard A. Cerione, Ithaca, NY (US)

(73) Assignee: CORNELL UNIVERSITY, Ithaca, NY (US)

(21) Appl. No.: 13/821,834

(22) PCT Filed: Sep. 12, 2011
(86) PCT No.: PCT/US11/51228

§ 371 Date: May 21, 2013

Related U.S. Application Data
(60) Provisional application No. 61/381,461, filed on Sep. 10, 2010.

(51) Int. Cl. A61K 31/473; A61K 31/277; A61K 31/436; A61K 31/515; G01N 33/68; C12Q 1/34

(52) U.S. Cl. CPC A61K 31/473; G01N 33/6893; C12Q 1/34; A61K 31/436; A61K 31/515; A61K 31/277
USPC 514/270; 435/375; 435/7.4; 435/18; 514/284; 506/10; 514/520; 514/291

(57) ABSTRACT

A method of reducing the production of glutamate from glutamine by glutaminase C in a cell or tissue. The method involves inhibiting activating phosphorylation of glutaminase C under conditions effective to reduce production of glutamate from glutamine. Methods for treating or preventing a condition mediated by the activating phosphorylation of glutaminase C, detecting a condition mediated by the activating phosphorylation of glutaminase C, and screening for compounds capable of treating or preventing cancer are also disclosed.

### CROSS REFERENCE TO RELATED APPLICATION

This application claims the benefit under 35 U.S.C. §119(e) of U.S. Provisional Patent Application No. 61/381,461 filed Sep. 10, 2010, and this provisional application is incorporated herein by reference in its entirety.

This invention was made with government support under grant numbers RO1 GM40654, RO1 GM47458, and RO1 GM61762 awarded by National Institutes of Health. The U.S. Government has certain rights in this invention.

### FIELD OF INVENTION

The present invention relates to the inhibition of glutaminase C (GAC).

### BACKGROUND OF THE INVENTION

Tumor cells have an absolute requirement for glutamine as a growth substrate. Glutamine is required as a precursor for both DNA synthesis and protein synthesis, and may also be used as a respiratory substrate. In experiments where glutamine metabolism in tumor cells has been specifically compared with that in non-transformed cells of the same origin, glutamine metabolism in the tumor cells has been found to be considerably faster. This is true for human hepatocytes and hepatoma cells (Souba, W., “Glutamine and Cancer,” Ann. Surg. 218:715-728 (1993)) and also for glutamine oxidation in rat kidney fibroblasts and rat fibrosarcoma cells (Fischer et al., “Adaptive Alterations in Cellular Metabolism and Malignant Transformation,” Ann. Surg. 227:627-634 (1998)).

The first reaction in glutamine metabolism is hydrolysis of glutamine to glutamate via the mitochondrial enzyme phosphate-dependent glutaminase. Two major isoforms of this enzyme have been characterized. These are known as the kidney form (K-type) which was first cloned from rat kidney (Shapiro et al., “Isolation, Characterization, and In vitro Expression of a cDNA That Encodes the Kidney Isoenzyme of the Mitochondrial Glutaminase,” J. Biol. Chem. 266:18792-18796 (1991)) and is expressed in many mammalian tissues, and the liver form (L-type) (Chung-Bok et al., “Rat Hepatic Glutaminase, Identification of the Full Coding Sequence and Characterization of a Functional Promoter,” Biochem. J. 324:193-200 (1997)) which was originally identified in post-natal liver. These two enzymes have different kinetic properties. Although the cDNAs encoding the two isoforms have regions of high sequence similarity, they also differ significantly elsewhere and the enzyme isoforms are the products of different genes (for a review see (Curthoys et al., “Regulation of Glutaminase Activity and Glutamine Metabolism,” Annu. Rev. Nutr. 16:133-159 (1995)). Glutamine metabolism is essential for tumor cell growth but there are few studies at present on glutaminase expression in tumor cells. In mouse Ehrlich ascites cells (Quesada et al., “Purification of Phosphate-Dependent Glutaminase from Isolated Mitochondria of Ehrlich Ascites-Tumor Cells,” Biochem. J. 255:1031-1035 (1988)) and rat fibrosarcoma cells (Fischer et al., “Adaptive Alterations in Cellular Metabolism and Malignant Transformation,” Ann. Surg. 227:627-634 (1998)), an enzyme with the kinetic properties of the K-type glutaminase is expressed. Rat and human hepatocytes express the L-type glutaminase, but this is not expressed in hepatoma cell lines, which express the K-type instead (Souba, W. W., “Glutamine and Cancer,” Ann. Surg. 218:715-728 (1993)). Inhibition of K-type glutaminase expression by anti-sense mRNA in Ehrlich ascites cells has been shown to decrease the growth and tumorigenicity of these cells (Lobo et al., “Inhibition of Glutaminase Expression by Antisense mRNA Decreases Growth and Tumorigenicity of Tumor Cells,” Biochem. J. 348:257-261 (2000)).

Glutaminase C is the isoform-2 of the human glutaminase, an enzyme found in kidney and other tissues and generally referred as kidney-type glutaminase. Glutaminase C is involved in the hydrolysis of glutamine to glutamate and ammonium. Generally, Glutaminase C is present in either an inactive state or an active state; however, little is known about the transition of Glutaminase C from the inactive state to the active state. The present invention is directed to overcoming these and other deficiencies in the art.

### SUMMARY OF THE INVENTION

A first aspect of the present invention relates to a method of reducing the production of glutamate from glutamine in a cell or a tissue comprising inhibiting activating phosphorylation within the amino acid sequence of SEQ ID NO: 1 of glutaminase C in the cell or tissue under conditions effective to reduce production of glutamate from glutamine.

In certain embodiments, the amino acid residue X of SEQ ID NO: 1 is valine or alanine. In certain embodiments, the activating phosphorylation event occurs at serine 95 of the amino acid sequence of SEQ ID NO: 2 or at serine 103 of the amino acid sequence of SEQ ID NO: 3.

In particular embodiments, the inhibition of the activating phosphorylation within the amino acid sequence of SEQ ID NO: 1 of glutaminase C is carried out by inhibiting a transcription factor which regulates phosphorylation of glutaminase C or a kinase which phosphorylates glutaminase C. In one embodiment, the kinase is mTOR. In a related embodiment, mTOR is inhibited by rapamycin or its homologs. In another embodiment, the transcription factor is NF-κB. In a related embodiment, NF-κB is inhibited by BAY11-7082.

A second aspect of the present invention relates to a method of treating or preventing a condition mediated by activating phosphorylation of glutaminase C in a subject. The method involves selecting a subject having or being susceptible to a condition mediated by activating phosphorylation within the amino acid sequence of SEQ ID NO: 1 of glutaminase C and administering to said selected subject an inhibitor of the activating phosphorylation within the amino acid sequence of SEQ ID NO: 1 of glutaminase C under conditions effective to treat or prevent the condition mediated by the activating phosphorylation of glutaminase C.

In certain embodiments, the amino acid residue X of SEQ ID NO: 1 is valine or alanine. In certain embodiments, the activating phosphorylation event occurs at serine 95 of the amino acid sequence of SEQ ID NO: 2 or at serine 103 of the amino acid sequence of SEQ ID NO: 3.

In particular embodiments, the inhibition of the activating phosphorylation within the amino acid sequence of SEQ ID NO: 1 of glutaminase C is carried out by inhibiting a transcription factor which regulates phosphorylation of glutaminase C or a kinase which phosphorylates glutaminase C. In one embodiment, the kinase is mTOR. In a related embodiment, mTOR is inhibited by rapamycin or its homologs or other inhibitors of mTOR. In another embodiment, the transcriptional factor is NF-κB. In a related embodiment, NF-κB is inhibited by BAY11-7082.

In certain embodiments, the condition is cancer. In one embodiment, the cancer is breast cancer, lung cancer, brain cancer, pancreatic cancer, or colon cancer.

A third aspect of the present invention relates to a method of detecting a condition mediated by activating phosphorylation within the amino acid sequence of SEQ ID NO: 1 of glutaminase C, wherein the method comprises the steps of providing a cell or tissue; providing a reagent that specifically recognizes the activating phosphorylation within the amino acid sequence of SEQ ID NO: 1 of glutaminase C; contacting the cell or tissue with the reagent under conditions effective for the reagent to bind to phosphorylated glutaminase C and form a phosphorylated glutaminase C-reagent conjugate; and identifying presence of the phosphorylated glutaminase C-reagent conjugate wherein the formation of the phosphorylated glutaminase C-reagent conjugate indicates the existence of a condition mediated by the activating phosphorylation of glutaminase C. In certain embodiments, the reagent is an antibody.

A fourth aspect of the present invention relates to a method screening for compounds capable of treating or preventing cancer, wherein the method comprises the steps of providing a cancer cell or cancer tissue under conditions effective to produce glutamate from glutamine as a result of glutaminase C activity; providing a plurality of candidate compounds; contacting the cancer cell or cancer tissue with the candidate compounds under conditions effective for activating phosphorylation; and identifying the candidate compounds which inhibit the activating phosphorylation of glutaminase C within the amino acid sequence of SEQ ID NO: 1 as a result of said contacting as having potential capability of treating or preventing cancer.

In certain embodiments, the screening method further comprises lysing the cancer cell or cancer tissue after said contacting and before said identifying, wherein said identifying involves using an antibody that binds to phosphorylated glutaminase C. In another embodiment, the cancer cell or cancer tissue is cultured in the presence of radiolabelled phosphate and the activating phosphorylation of glutaminase C is determined by detecting radiolabelled glutaminase C.

### BRIEF DESCRIPTION OF THE DRAWINGS

FIGS. 1A-E illustrate that the small molecule 968 inhibits cellular transformation. FIG. 1A shows NIH 3T3 cells that are transiently transfected with oncogenic Dbl and cultured for 14 days, while treated with different benzo[a]phenanthridinones (designated 384, 335, 968, 537, and 343) (10 μM each). Cells were fixed and stained with crystal violet for counting of foci. Right: 968 was serially diluted (10, 5, 2.5, and 1.25 μM) and evaluated for its ability to inhibit focus formation. FIG. 1B shows NIH 3T3 cells that are stably transfected with Dbl and grown in DMEM supplemented with 1% calf serum and the indicated amounts of 968 or BA-968. After 6 days, the cells were counted. 100% represents the number of Dbl-transformed cells counted in the absence of 968 (27.5×10⁴ cells). Data represent the average of 3 experiments (±s.d.). FIG. 1C shows the different benzo[a]phenanthridinone derivatives examined for their effects on Dbl-induced focus formation (designated 968, BA968, 335, 343, 031, 537, 5043, and 384). FIG. 1D shows control NIH 3T3 cells that were cultured in DMEM supplemented with 10% calf serum, and either untreated or treated with 10 μM 968 or 335. At the indicated times, the cells were counted. Data represent the average of 3 experiments (±s.d.). FIG. 1E shows photomicrographs of Dbl-transfected NIH 3T3 cells (bottom panels) and control NIH 3T3 cells (top panels) treated with either DMSO or 5 μM 968.

FIGS. 2A-G illustrate effects of 968 on the transforming activity of constitutively active Rho GTPases and human breast cancer cells. FIG. 2A (top) shows NIH 3T3 cells stably expressing hemagglutinin (HA)-tagged Cdc42(F28L), Rac(F28L), RhoC(F30L), or vector control cells, either treated with 10 μM 968 or untreated, grown in soft agar. Cells were scored after 14 days and plotted as the percentage of the total number of colonies greater than 50 μm in diameter. Data represent the average of 3 experiments (±s.d.). FIG. 2A (bottom) shows the relative expression of the HA-tagged GTPases. FIG. 2B shows cells that were treated with 10 μM 968 or untreated, cultured in DMEM supplemented with 10% calf serum for 6 days, and then counted. Data represent the average of 3 experiments (±s.d.). FIG. 2C shows cells that were cultured in DMEM supplemented with 1% calf serum, treated with 10 μM 968 or untreated, and counted at the indicated times. Data represent the average of 3 experiments (±s.d.). FIG. 2D shows cells that were serum-starved, treated with 10 μM 968 or untreated, and seeded in MilliCell upper chambers containing growth factor-reduced Matri-gel. After 24 hours at 37° C., the migratory cells were fixed, stained with GIEMSA, and counted. Data represent the average of 3 experiments (±s.d.). FIG. 2E shows MDA-MB231 cells, SKBR3 cells, and NIH 3T3 cells stably expressing Dbl that were treated with 10 μM 968 or untreated, and grown in soft-agar as in FIG. 2A. Data represent the average of 3 experiments (±s.d.). FIG. 2F shows breast cancer cells that were cultured in RPMI 1640 medium supplemented with 10% fetal bovine serum, and HMECs were cultured in MEGM complete medium, for 6 days in the presence or absence of 10 μM 968, and then counted. Data represent the average of 3 experiments (±s.d.). FIG. 2G shows breast cancer cells that were cultured in RPMI 1640 medium supplemented with 1% fetal bovine serum, treated with 10 μM 968 or untreated, and analyzed as in 2C. Data represent the average of 3 experiments (±s.d.).

FIGS. 3A-G show that glutaminase C serves as a target for 968. FIG. 3A shows the E. coli-expressed mouse ortholog of human GAC that was assayed in the presence or absence of the indicated amounts of 968 () or 335 (◯). 100%=620 moles of glutamine hydrolyzed per minute per mole of enzyme. Data represent the average of 3 experiments (±s.d.). FIG. 3A (top panel) shows the biotin-labeled, active moiety of 968 linked to streptavidin-agarose beads, or control beads alone that was incubated with NIH 3T3 cell lysates transiently expressing VS-tagged mouse GAC. Following precipitation of the beads and re-suspension, the samples were analyzed by Western blotting with anti-V5 antibody. FIG. 3B (left) shows NIH 3T3 cells stably expressing HA-Cdc42(F28L), cells stably expressing HA-Cdc42(F28L) transfected with control siRNA or siRNAs targeting both isoforms of mouse KGA, or control cells that are grown in DMEM supplemented with 1% calf serum and counted. Data represent the average of 3 experiments (±s.d.). FIG. 3B (right) shows the efficiencies of siRNAs targeting both isoforms of KGA, and the relative levels of HA-Cdc42 in the different cells. FIG. 3C (top) shows breast cancer cells that were grown in RPMI 1640 medium supplemented with 1% fetal bovine calf serum. Data represent the average of 3 experiments (±s.d.). FIG. 3C (bottom) shows the relative efficiencies of siRNAs targeting both isoforms of KGA. FIG. 3D shows SKBR3 cells that were grown in 1% fetal bovine serum as in 3C except in the presence of 10 μM 968 alone or together with 7 mM dimethyl α-ketoglutarate. Data represent the average of 3 experiments (±s.d.). FIG. 3E NIH 3T3 cells transiently expressing Dbl were assayed for focus formation in the presence of 10 μM 968 alone or together with 7 mM dimethyl α-ketoglutarate. FIG. 3F (top) shows mitochondrial fractions from equivalent numbers of the indicated stable cell lines which had been treated with or without 10 μM 968 for 48 hours and then assayed for basal (phosphate-independent) GA activity. 100%=680 nM glutamine hydrolyzed per minute per 10⁵ cells. Data are the average of 3 experiments (±s.d.). FIG. 3G (bottom) shows the relative amounts of KGA (using an antibody which recognizes both isoforms) in the mitochondrial preparations. FIG. 3G (top) shows the mitochondrial fractions from equivalent numbers of the indicated cells which had been treated with or without 10 μM 968 for 48 hours and then assayed for basal (phosphate-independent) GA activity. 100%=750 nM glutamine hydrolyzed per minute per 10⁵ cells. Data represent the average of 3 experiments (±s.d.). FIG. 3G (bottom) shows the relative amounts of KGA and VDAC present in the mitochondrial preparations.

FIGS. 4A-E illustrate the role of glutaminase C activity in cellular transformation. FIG. 4A (left) shows control NIH 3T3 cells, NIH 3T3 cells transiently expressing Dbl, cells stably expressing Cdc42(F28L) and transiently expressing mouse GAC, cells stably expressing Cdc42(F28L), cells transiently expressing GAC alone, and cells stably expressing Cdc42(F28L) and transiently expressing Dbl that were examined for focus-forming activity. FIG. 4A (right) shows the quantification of foci. Data represent the average of 3 experiments (±s.d.). FIG. 4B (left) shows the focus-forming assays performed on NIH 3T3 cells stably expressing Cdc42(F28L), cells stably expressing Cdc42(F28L) and transiently expressing Dbl, and cells stably expressing Cdc42(F28L) and either transiently expressing wild-type mouse GAC or the GAC(S291A) mutant. FIG. 4B (right) shows the quantification of foci. Data represent the average of 3 experiments (±s.d.). FIG. 4C (top) shows the basal (phosphate-independent) GA activity in the mitochondrial fractions from NIH 3T3 cells stably expressing Dbl that were cultured for 2 days and treated or untreated with 2 μM BAY 11-7082, or transfected with control siRNA or siRNAs targeting the p65/RelA subunit. 100% represents the activity measured in untreated Dbl-transformed cells. The data represent the average of 2 experiments. FIG. 4C (bottom) shows the relative amounts of KGA (using an antibody which recognizes both isoforms) present in the mitochondria from the indicated cells, and the relative efficiencies of two siRNAs targeting p65/RelA. FIG. 4D (top) shows the basal (phosphate-independent) GA activity in the mitochondrial fractions from HMECs, and SKBR3 cells treated or untreated with 2 μM BAY 11-7082, or transfected with siRNAs targeting p65/RelA. 100% represents the activity measured in untreated SKBR3 cells. The data represent the average of 2 experiments. FIG. 4D (bottom) shows the relative amounts of KGA (using an antibody which recognizes both isoforms) present in the mitochondria from the indicated cells, and the relative efficiencies of two siRNAs targeting p65/RelA. FIG. 4E (top) shows that V5-GAC was transiently expressed in NIH 3T3 cells stably expressing Dbl that were treated with 2 μM BAY 11-7082, 10 μM 968, 10 μM BA-968 or untreated, or in control NIH 3T3 cells, and then immunoprecipitated and assayed for GA activity in the absence of phosphate. Data represent the average of 3 experiments (±s.d.). FIG. 4E (bottom) shows the relative expression of V5-GAC.

FIGS. 5A-C illustrate comparative abilities of 968 and other benzo[a]phenanthridinones to inhibit the transforming activity of oncogenic Dbl and H-Ras. FIG. 5A shows NIH 313 cells that were transiently transfected with oncogenic Dbl for 14 days while treated with different benzo[a]phenanthridinones (designated 384, 335, 968, 537, 343, 031, and 5043, see FIG. 1C for structures) (5 μM, each) that were dissolved in DMSO. Histograms show the relative levels of Dbl-induced focus formation for the different treatments, compared to Dbl-induced focus formation measured in the presence of DMSO (i.e. solvent control). Data represent the average of 3 experiments (mean±s.d.). FIG. 5B shows NIH 3T3 cells that were transiently transfected with H-Ras(G12V) and cultured for 14 days, while treated with different benzo[a}phenanthridinones (5 μM, each) that were dissolved in DMSO. Histograms show the relative levels of Ras(G12V)-induced focus formation measured for the different treatments, compared to Ras(G12V)-induced focus formation measured in the presence of DMSO (solvent control). Data represent the average of 3 experiments (mean±s.d.). FIG. 5C NIH 3T3 cells stably expressing H-Ras(G12V) that were cultured in DMEM supplemented with 1% calf serum, with the indicated amounts of 968. After 6 days, cells are trypsinized and counted. 100% represents the number of cells counted in the absence of 968, i.e. 26×10⁴ cells. Data represent the average of 3 experiments (mean±s.d.).

FIG. 6 shows that Rho GTPases are hyper-activated in breast cancer cells. Lysates from MDA-MB231 cells, SKBR3 cells, and HMECs, were prepared and incubated with GST fused to the limit Rho-binding domain on Rhotekin (GST-RBD). The top panels show the relative levels of RhoA-GTP and RhoC-GTP that were co-precipitated with GST-RBD from the indicated cells, as indicated by Western blotting with an anti-RhoA monoclonal antibody and an anti-RhoC polyclonal antibody. The middle panels compare the relative expression of RhoA and RhoC in whole cell lysates (WCL) from the different cells and the bottom panel shows the relative input of GST-RBD.

FIG. 7 illustrates the MS peptide analysis of the silver-stained band that was specifically precipitated by the biotin-labeled, active moiety of 968. Shown in the figure is the alignment of mouse kidney-type glutaminase (KGA) isoform-1 (SEQ ID NO: 4) and isoform-2 (the mouse ortholog of human GAC) (SEQ ID NO: 3). The peptides identified by the Maldi-MS analysis of the protein precipitated by the biotin-labeled, active moiety of 968 are in bold and underlined. The peptide VLSPEAVR (SEQ ID NO: 5) is present in both isoforms whereas VSPESSDDTSTTVVYR (SEQ ID NO: 6) maps to the C-terminus unique to the mouse GAC (Accession # NP_{—}001106854). GAC has a predicted molecular weight of 65,864.

FIG. 8 illustrates that the biotin-labeled, active moiety of 968 binds to a 66 kDa protein that cross-reacts with the anti-KGA polyclonal antibody. The biotin-labeled, active moiety of 968 linked to streptavidin-agarose beads, or control beads alone, were incubated with lysates from NIH 3T3 cells stably expressing the constitutively active Cdc42(F28L) mutant. Following precipitation of the beads and re-suspension, the samples are analyzed by SDS-PAGE and silver-staining (left-hand panel), as well as by Western blot analysis using an anti-KGA polyclonal antibody.

FIGS. 9A-C show that 968 is not competitive versus either the GA-substrate, glutamine, nor inorganic phosphate, an allosteric activator, of GA activity. The activity of the E. coli-expressed recombinant mouse ortholog of human GAC were assayed in the presence of 0 (), 10 (∇) or 20 (▪) μM 968 and inorganic phosphate in the form of dipotassium hydrogen phosphate. FIG. 9A shows that the concentration of inorganic phosphate is kept constant at 150 mM and the concentration of glutamine was varied from 0 to 50 mM. FIG. 9B shows the data in FIG. 9A shown as a double-reciprocal lineweaver-burke plot. FIG. 9C shows that the concentration of glutamine is kept constant at 20 mM and the concentration of inorganic phosphate was varied from 0 to 200 mM. The data are plotted as GA activity as function of varying concentrations of glutamine or inorganic phosphate. The data are the average of 3 experiments and are plotted as mean±SEM.

FIGS. 10A-C illustrate the effects of knocking-down KGA on the growth of transformed/cancer cells versus NIH 3T3 cells. FIG. 10A shows NIH 313 cells stably expressing Cdc42(F28L), cells stably expressing Cdc42(F28L) transfected with control siRNA or siRNAs targeting both isoforms of mouse KGA, and control (vector) cells, grown in soft agar and scored after 10 days. Histograms show the percentage of the total number of colonies greater than 50 μm in diameter. Data represent the average of 3 experiments (mean±s.d.). FIG. 10B shows NIH 3T3 cells transfected with control siRNA or siRNAs targeting both isoforms of mouse KGA, cultured in DMEM supplemented with 10% calf serum for the indicated number of days, and then trypsinized and counted. FIG. 10B (top) shows histograms that represent the average of 3 experiments (mean±s.d.). FIG. 10B (bottom panel) shows the relative efficiencies of the siRNAs targeting KGA. FIG. 10C shows the indicated breast cancer cell lines transfected with control siRNA or with siRNAs targeting both isoforms of KGA and then grown in soft agar and scored after 10 days as described in S6A. Data represent the average of 3 experiments (mean±s.d.).

FIGS. 11A-E illustrate the effects of different treatments on GA activity. FIG. 11A shows mitochondrial fractions from equivalent numbers of the indicated stable cell lines which had been treated with or without 10 μM 968 for 48 hours and then assayed for basal (phosphate-independent) GA activity in the presence of 133 mM inorganic phosphate (+Pi). The addition of Pi stimulated the GA activity in control 3T3 cells, Dbl-expressing cells, and Cdc42(F28L)-expressing cells by ˜6-fold, 2-fold, and 3-fold, respectively. 100% represents the Pi-stimulated activity measured for Dbl-transformed cells that were not treated with 968. Data is the average of 3 experiments (±s.d.). FIG. 11B shows mitochondrial fractions from equivalent numbers of the indicated cells, treated or untreated with 10 μM 968, assayed for GA activity in the presence of 133 mM Pi. The addition of Pi stimulated the GA activity of HMECs, MDA-MB231 cells, and SKBR3 cells by ˜5-fold, 2-fold, and 1.4-fold, respectively. 100% represents the Pi-stimulated activity for SKBR3 cells that were not treated with 968. Data are the average of 3 experiments (±s.d.). FIG. 11C (left) shows that SKBR3 cells are transfected with control siRNA or siRNAs targeting the RhoA and RhoC GTPases (i.e. a double knock-down). Mitochondrial fractions were prepared from equal numbers of cells and assayed for GA activity in the presence or absence of 133 mM Pi. The data are plotted as the percentage of GA activity measured for untreated SKBR3 cells and represent the average of 2 experiments. FIG. 11C (right) shows that the efficiencies of the siRNAs against RhoC and RhoA were assessed by Western blot analysis using anti-RhoA and anti-RhoC antibodies. FIG. 11D shows Pi-stimulated GA activity in mitochondrial fractions from NIH 3T3 cells stably expressing Dbl that were cultured for 2 days and treated or untreated with 2 μM BAY 11-7082, or transfected with control siRNA or siRNAs targeting the p65/RelA subunit. 100% represents the Pi-stimulated activity measured for untreated Dbl-transformed cells. The data represent the average of 2 experiments. FIG. 11E shows Pi-stimulated GA activity in the mitochondrial fractions from SKBR3 cells treated with 2 μM BAY 11-7082, or transfected with control siRNA or siRNAs targeting p65/RelA. 100% represents the Pi-stimulated activity for untreated SKBR3 cells. The data represent the average of 2 experiments.

FIG. 12 illustrates GAC expression levels in normal and cancerous breast tissues obtained from 80 patients.

FIG. 13 shows that GAC, but not KGA, mRNA levels are increased in higher grade breast tumors.

FIG. 14 shows that GAC, but not KGA, enhances the oncogenic potential of Cdc42.

FIG. 15 illustrates that GAC is differentially phosphorylated in transformed (Dbl) cells but not in normal NIH 3T3 cells.

FIG. 16 illustrates that the phosphorylation of GAC is necessary for its basal glutaminase activity.

FIG. 17 illustrates that treatment of cells with 968 inhibits the formation of at least one phosphorylation on GAC.

FIG. 18 illustrates a model for the mode of action of 968 on GAC and oncogenic growth.

FIG. 19 illustrates that both 968 and BA-968 were effective inhibitors of GAC activity.

FIG. 20 illustrates the chemical structure of the NF-kB inhibitor Bay 11-7082.

FIG. 21 shows the 2D gel of GAC expressed by normal NIH 3T3 cells (first panel) or Dbl-transformed NIH 3T3 cells (second to fifth panels). The most negative major species of GAC is indicated with an arrow.

FIG. 22 shows a mass spectrum of the most negative major species of a VS-tagged GAC expressed by Dbl-transfected NIH 3T3 cells that indicates that serine 103 of mouse GAC (SEQ ID NO: 3) was phosphorylated.

FIG. 23 shows the isolated peptide GGTPPQQQQQQQQQPGAS*PPAAPGPK (SEQ ID NO: 7), wherein the serine residue corresponds to Serine 95 of the human GAC sequence (SEQ ID NO: 2) and Serine 103 of the mouse GAC sequence (SEQ ID NO: 3).

FIG. 24 is a bar graph that shows the effect of small molecule 968 on the glutaminase activity of wild-type GAC, a C-terminal truncation of GAC that has impaired glutaminase activity (GACΔC), and a phospho-defective GAC mutant (GAC S103A).

FIG. 25 shows the effects of small molecule 968, NF-kB inhibitor Bay 11-7082, and mTOR inhibitor rapamycin on mitochondrial glutaminase activity. Dbl expressing NIH 3T3 cells were treated for 48 hours in the presence or absence of 968 (10 μM), Bay 11-7082 (2 μM) or Rapamycin (100 nM). Mitochondria were then isolated from the treated cells and assayed for glutaminase activity in the absence or presence of phosphate.

### DETAILED DESCRIPTION OF THE INVENTION

As used in this specification and the appended claims, the singular forms “a,” “an” and “the” include plural references unless the content clearly dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Each embodiment in this specification is to be applied mutatis mutandis to every other embodiment unless expressly stated otherwise.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

As used above, and throughout the description of this invention, the following terms, unless otherwise indicated, shall be understood to have the following meanings. If not defined otherwise herein, all technical and scientific terms used herein have the same meaning as is commonly understood by one of ordinary skill in the art to which this invention belongs. In the event that there is a plurality of definitions for a term herein, those in this section prevail unless stated otherwise.

The term “halo” or “halogen” means fluoro, chloro, bromo, or iodo.

The term “optionally substituted” indicates that a group may have a substituent at each substitutable atom of the group (including more than one substituent on a single atom), and the identity of each substituent is independent of the others.

The term “substituted” or “substitution” of an atom means that one or more hydrogen on the designated atom is replaced with a selection from the indicated group, provided that the designated atom's normal valency is not exceeded. “Unsubstituted” atoms bear all of the hydrogen atoms dictated by their valency. When a substituent is oxo (i.e., ═O), then 2 hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent. Exemplary substitutents include, without limitation, oxo, thio (i.e. ═S), nitro, cyano, halo, OH, NH₂, C₁-C₆ alkyl, C₁-C₆ alkoxy, C₂-C₆ alkenyl, C₂-C₆ alkynyl, C₃-C₆ cycloalkyl, C₄-C₇ cycloalkylalkyl, monocyclic aryl, monocyclic hetereoaryl, polycyclic aryl, and polycyclic heteroaryl.

The term “monocyclic” indicates a molecular structure having one ring.

The term “polycyclic” indicates a molecular structure having two or more rings, including, but not limited to, fused, bridged, or spiro rings.

The term “alkyl” means an aliphatic hydrocarbon group which may be straight or branched having about 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

The term “alkenyl” means an aliphatic hydrocarbon group containing a carbon-carbon double bond and which may be straight or branched having about 2 to about 6 carbon atoms in the chain. Preferred alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl.

The term “alkynyl” means an aliphatic hydrocarbon group containing a carbon-carbon triple bond and which may be straight or branched having about 2 to about 6 carbon atoms in the chain. Preferred alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl.

The term “alkoxy” means an alkyl-O—, alkenyl-O—, or alkynyl-O— group wherein the alkyl, alkenyl, or alkynyl group is described above. Exemplary alkoxy groups include methoxy, ethoxy, n-propoxy, i-propoxy, n-butoxy, pentoxy, and hexoxy.

The term “cycloalkyl” refers to a non-aromatic saturated or unsaturated mono- or polycyclic ring system which may contain 3 to 6 carbon atoms; and which may include at least one double bond. Exemplary cycloalkyl groups include, without limitation, cyclopropyl, cyclobutyl, cyclopentyl, cyclohexyl, cyclopropenyl, cyclobutenyl, cyclopentenyl, cyclohexenyl, anti-bicyclopropane, or syn-bicyclopropane.

The term “cycloalkylalkyl” refers to a radical of the formula —R^{a}R^{b} where R^{a} is an alkyl radical as defined above and R^{b} is a cycloalkyl radical as defined above. The alkyl radical and the cycloalkyl radical may be optionally substituted as defined above.

The term “aryl” refers to aromatic monocyclic or polycyclic ring system containing from 6 to 19 carbon atoms, where the ring system may be optionally substituted. Aryl groups of the present invention include, but are not limited to, groups such as phenyl, naphthyl, azulenyl, phenanthrenyl, anthracenyl, fluorenyl, pyrenyl, triphenylenyl, chrysenyl, and naphthacenyl.

The term “arylalkyl” refers to a radical of the formula —R^{a}R^{b} where R^{a} is an alkyl radical as defined above and R^{b} is an aryl radical as defined above. The alkyl radical and the cycloalkyl radical may be optionally substituted as defined above.

The term “aryarylalkyl” refers to a radical of the formula —R^{a}R^{b}R^{c} where R^{a} is an alkyl as defined above, R^{b} is an aryl radical as defined above, and R^{c} is an aryl radical as defined above. The alkyl radical and both aryl radicals may be optionally substituted as defined above.

The term “heterocyclyl” refers to a stable 3- to 18-membered ring radical which consists of carbon atoms and from one to five heteroatoms selected from the group consisting of nitrogen, oxygen and sulfur. For purposes of this invention, the heterocyclyl radical may be a monocyclic, or a polycyclic ring system, which may include fused, bridged, or spiro ring systems; and the nitrogen, carbon, or sulfur atoms in the heterocyclyl radical may be optionally oxidized; the nitrogen atom may be optionally quaternized; and the ring radical may be partially or fully saturated. Examples of such heterocyclyl radicals include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone.

The term “heteroaryl” refers to an aromatic ring radical which consists of carbon atoms and from one to five heteroatoms selected from the group consisting of nitrogen, oxygen, and sulfur. For purposes of this invention the heteroaryl may be a monocyclic or polycyclic ring system; and the nitrogen, carbon, and sulfur atoms in the heteroaryl ring may be optionally oxidized; the nitrogen may optionally be quaternized. Examples of heteroaryl groups include, without limitation, pyrrolyl, pyrazolyl, imidazolyl, triazolyl, furyl, thiophenyl, oxazolyl, isoxazolyl, thiazolyl, isothiazolyl, oxadiazolyl, thiadiazolyl, pyridyl, pyrazinyl, pyrimidinyl, pyridazinyl, triazinyl, thienopyrrolyl, furopyrrolyl, indolyl, azaindolyl, isoindolyl, indolinyl, indolizinyl, indazolyl, benzimidazolyl, imidazopyridinyl, benzotriazolyl, benzoxazolyl, benzoxadiazolyl, benzothiazolyl, pyrazolopyridinyl, triazolopyridinyl, thienopyridinyl, benzothiadiazolyl, benzofuyl, benzothiophenyl, quinolinyl, isoquinolinyl, tetrahydroquinolyl, tetrahydroisoquinolyl, cinnolinyl, quinazolinyl, quinolizilinyl, phthalazinyl, benzotriazinyl, chromenyl, naphthyridinyl, acrydinyl, phenanzinyl, phenothiazinyl, phenoxazinyl, pteridinyl, and purinyl.

Further heterocycles and heteraryls are described in Katritzky et al., eds., “Comprehensive Heterocyclic Chemistry: The Structure, Reactions, Synthesis and Use of Heterocyclic Compounds,” Vol. 1-8, Pergamon Press, N.Y. (1984), which is hereby incorporated by reference in its entirety.

The term “compounds of the present invention”, and equivalent expressions are meant to embrace compounds of general Formulae (I), (II), and/or (III) (as well as compounds comprising their active moieties) as herein before described, which expression includes the prodrugs, the pharmaceutically acceptable salts, and the solvates, e.g., hydrates, where the context so permits. Similarly, reference to intermediates, whether or not they themselves are claimed, is meant to embrace their salts and solvates, where the context so permits. For the sake of clarity, particular instances, when the context so permits, are sometimes indicated in the text, but these instances are purely illustrative and it is not intended to exclude other instances when the context so permits.

This invention also envisions the “quaternization” of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

The terms “polypeptide,” “protein” and “peptide” are used interchangeably and mean a polymer of amino acids not limited to any particular length. The term does not exclude modifications such as myristylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence.

The term “isolated protein” referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein with which the “isolated protein” is associated in nature, (6) is operably associated (by covalent or noncovalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

The term “polypeptide fragment” refers to a polypeptide, which can be monomeric or multimeric, that has an amino-terminal deletion, a carboxyl-terminal deletion, and/or an internal deletion or substitution of a naturally-occurring or recombinantly-produced polypeptide.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single and double stranded forms of DNA.

The term “isolated polynucleotide” as used herein shall mean a polynucleotide of genomic, cDNA, or synthetic origin or some combination thereof, which by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, Oligonucleotides and Analogues: A Practical Approach, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby incorporated by reference for any purpose. An oligonucleotide can include a detectable label to enable detection of the oligonucleotide or hybridization thereof.

The term “vector” is used to refer to any molecule (e.g., nucleic acid, plasmid, or virus) used to transfer a polynucleotide sequence to a host cell. The term “expression vector” refers to a vector that is suitable for transformation of a host cell and contains nucleic acid sequences that direct and/or control expression of inserted nucleic acid sequences. Expression includes, but is not limited to, processes such as transcription, translation, and RNA splicing, if introns are present.

As will be understood by those skilled in the art, polynucleotides may include genomic sequences, extra-genomic and plasmid-encoded sequences and smaller engineered gene segments that express, or may be adapted to express, proteins, polypeptides, peptides and the like. Such segments may be naturally isolated, or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence that encodes a variant or derivative of such a sequence.

“Carriers” as used herein include pharmaceutically or physiologically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

As used herein, “treating” or “treatment” refers to an approach for obtaining beneficial or desired results, including and preferably clinical results. Treatment can involve optionally either the amelioration of symptoms of the disease or condition, or the delaying of the progression of the disease or condition.

As used herein, unless the context makes clear otherwise, “prevention,” and similar words such as “prevented,” “preventing” etc., indicates an approach for preventing, inhibiting, or reducing the likelihood of, the onset or recurrence of a disease or condition. It also refers to preventing, inhibiting, or reducing the likelihood of, the occurrence or recurrence of the symptoms of a disease or condition, or optionally an approach for delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, “inhibiting cell growth” or “inhibiting proliferation of cells” refers to reducing or halting the growth rate of cells. For example, by inhibiting the growth of tumor cells, the rate of increase in size of the tumor may slow. In other embodiments, the tumor may stay the same size or decrease in size, i.e., regress. In particular embodiments, the rate of cell growth or cell proliferation is inhibited by at least 20%, at least 30%, at least 40%, at least 50%, at least 60%, at least 70%, at least 80%, or at least 90%.

The term “antibody” (Ab) as used herein includes monoclonal antibodies, polyclonal antibodies, multispecific antibodies (e.g., bispecific antibodies), and antibody fragments, so long as they exhibit the desired biological activity, e.g., specifically bind to Glutaminase C. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody is purified: (1) to greater than 95% by weight of antibody as determined by the Bradford method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

An “intact” antibody is one that comprises an antigen-binding site as well as a C_{L} and at least heavy chain constant domains, C_{H} 1, C_{H} 2 and C_{H} 3. The constant domains may be native sequence constant domains (e.g., human native sequence constant domains) or amino acid sequence variants thereof. Preferably, the intact antibody has one or more effector functions.

An “antibody fragment” is a polypeptide comprising or consisting of a portion of an intact antibody, preferably the antigen-binding or variable region of the intact antibody. Examples of antibody fragments include Fab, Fab′, F(ab′)₂, and Fv fragments; diabodies; linear antibodies (see U.S. Pat. No. 5,641,870; Zapata et al., Protein Eng. 8(10): 1057-1062 [1995], which are hereby incorporated by reference in their entirety); single-chain antibody molecules; and multispecific antibodies formed from antibody fragments. In particular, an “antigen-binding fragment” is a polypeptide comprising a portion of an intact antibody that specifically binds the target antigen, e.g., Glutaminase C.

As used herein, an antibody is said to be “immunospecific,” “specific for” or to “specifically bind” an antigen if it reacts at a detectable level with the antigen, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and in certain embodiments, a glutaminase C-specific antibody specifically binds to glutaminase C if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of antibodies can be readily determined using conventional techniques, for example, those described by Scatchard et al. (Ann. N.Y. Acad. Sci. USA 51:660 (1949), which is hereby incorporated by reference in its entirety).

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

As used herein, the term “activating phosphorylation” refers to either the phosphorylation of a protein or the phosphorylation state of a protein, such as glutaminase C, associated with an active conformation. For example, glutaminase C actively converts glutamine to glutamate when the serine at residue 103 of SEQ ID NO: 3 is phosphorylated, i.e., an activating phosphorylation.

As used herein, “active glutaminase C” or “active GAC” refers to a glutaminase C peptide that is in an active state or conformation. For example, an active glutaminase C is one that has been phosphorylated at the active phosphorylation site. An active glutaminase C may also be a mutated glutaminase C that is in a constitutively active conformation (e.g., S103A).

Glutaminase C is the isoform-2 of the human glutaminase, an enzyme found in kidney and other tissues and generally referred as kidney-type glutaminase. Glutaminase C is involved in the hydrolysis of glutamine to glutamate and ammonium.

Embodiments of the present invention relate in pertinent part to the surprising discovery that amino acid residue 103 of the mouse glutaminase C amino acid sequence set forth in SEQ ID NO: 3 (corresponding to amino acid residue 95 of the human glutaminase C amino acid sequence set forth in SEQ ID NO: 2) is important for the activating phosphorylation of glutaminase C. In particular, phosphorylation of Serine 103 of glutaminase C by the kinase mTOR was found to cause the transition of glutaminase C to its active conformation as described below. The present disclosure relates to methods of inhibiting the activating phosphorylation of glutaminase C.

### Inhibiting the Activating Phosphorylation of Glutaminase C

A first aspect of the present invention relates to a method of reducing the production of glutamate from glutamine in a cell or a tissue. The method involves inhibiting the activating phosphorylation of glutaminase C in the cell or tissue under conditions effective to reduce production of glutamate from glutamine. In particular, the activating phosphorylation that is inhibited occurs in the amino acid sequence set forth in SEQ ID NO: 1. In certain embodiments, the amino acid residue X of SEQ ID NO: 1 is valine or alanine. In certain embodiments, the activating phosphorylation event occurs at serine 95 of SEQ ID NO: 2 or at serine 103 of SEQ ID NO: 3.

The amino acid sequence of SEQ ID NO: 1 (consensus sequence) is as follows:

QPGXSPPAAP, where “X” can be any amino acid.

Human Glutaminase C (SEQ ID NO: 2) has the following amino acid sequence (the activating site is in bold and underlined):

```
    1 MMRLRGSGML RDLLLRSPAG VSATLRRAQP LVTLCRRPRG GGRPAAGPAA
  51 AARLHPWWGG GGWPAEPLAR GLSSSPSEIL QELGKGSTHP QPGVSPPAAP
101 AAPGPKDGPG ETDAFGNSEG KELVASGENK IKQGLLPSLE DLLFYTIAEG
151 QEKIPVHKFI TALKSTGLRT SDPRLKECMD MLRLTLQTTS DGVMLDKDLF
201 KKCVQSNIVL LTQAFRRKFV IPDFMSFTSH IDELYESAKK QSGGKVADYI
251 PQLAKFSPDL WGVSVCTADG QRHSTGDTKV PFCLQSCVKP LKYAIAVNDL
301 GTEYVHRYVG KEPSGLRFNK LFLNEDDKPH NPMVNAGAIV VTSLIKQGVN
351 NAEKFDYVMQ FLNKMAGNEY VGFSNATFQS ERESGDRNFA IGYYLKEKKC
401 FPEGTDMVGI LDFYFQLCSI EVTCESASVM AATLANGGFC PITGERVLSP
451 EAVRNTLSLM HSCGMYDFSG QFAFHVGLPA KSGVAGGILL VVPNVMGMMC
501 WSPPLDKMGN SVKGIHFCHD LVSLCNFHNY DNLRHFAKKL DPRREGGDQR
551 HSFGPLDYES LQQELALKET VWKKVSPESN EDISTTVVYR MESLGEKS
```

Mouse Glutaminase C (SEQ ID NO: 3) has the following amino acid sequence (the activating site is in bold and underlined):

```
    1 MMRLRGSAML RELLLRPPAA VGAVLRRAQP LGTLCRRPRG GSRPTAGLVA
  51 AARLHPWWGG GGRAKGPGAG GLSSSPSEIL QELGKGGTPP QQQQQQQQQP
101 GASPPAAPGP KDSPGETDAF GNSEGKEMVA AGDNKIKQGL LPSLEDLLFY
151 TIAEGQEKIP VHKFITALKS TGLRTSDPRL KECMDMLRLT LQTTSDGVML
201 DKDLFKKCVQ SNIVLLTQAF RRKFVIPDFM SFTSHIDELY ESAKKQSGGK
251 VADYIPQLAK FSPDLWGVSV CTVDGQRHSI GDTKVPFCLQ SCVKPLKYAI
301 AVNDLGTEYV HRYVGKEPSG LRFNKLFLNE DDKPHNPMVN AGAIVVTSLI
351 KQGVNNAEKF DYVMQFLNKM AGNEYVGFSN ATFQSERESG DRNFAIGYYL
401 KEKKCFPEGT DMVGILDFYF QLCSIEVTCE SASVMAATLA NGGFCPITGE
451 RVLSPEAVRN TLSLMHSCGM YDFSGQFAFH VGLPAKSGVA GGILLVVPNV
501 MGMMCWSPPL DKMGNSVKGI HFCHDLVSLC NFHNYDNLRH FAKKLDPRRE
551 GGDQRHSFGP LDYESLQQEL ALKDTVWKKV SPESSDDTST TVVYRMESLG
601 ERS
```

In certain embodiments, activating phosphorylation of glutaminase C is inhibited by inhibiting or blocking a kinase that phosphorylates glutaminase C, such as, mammalian target of rapamycin (mTOR). Inhibitors of mTOR include, for example, rapamycin, rapamycin derivatives or rapalogues, everolimus, sirolimus, and temsirolimus.

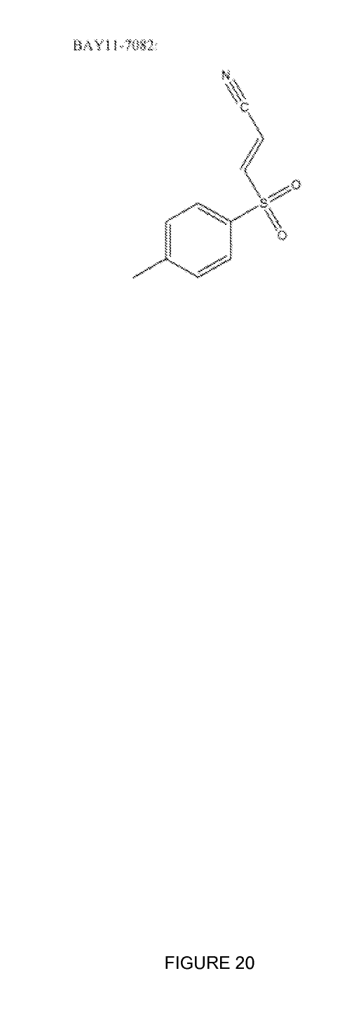

In certain embodiments, activating phosphorylation of glutaminase C is inhibited by inhibiting or blocking a transcription factor that regulates phosphorylation of glutaminase C, such as NF-κB Inhibitors of NF-κB include, for example, BAY 11-7082 (FIG. 20, E3((4-t-butylphenyl)-sulfonyl)-2-propenenitrile), Anethole, Anti-thrombin III, Azidothymidine, Benzyl isothiocyanate, cyanidin 3-O-glucoside, cyanidin 3-O-(2(G)-xylosylrutinoside), cyanidin 3-O-rutinoside, Buddlejasaponin IV, Cacospongionolide B, Calagualine, Carboplatin, Cardamonin, Cordycepin, Cycloepoxydon; 1-hydroxy-2-hydroxymethyl-3-pent-1-enylbenzene, Decursin, Delphinidin, Dexanabinol, Digitoxin, Docosahexaenoic acid, Gabexate mesilate, Gleevec (Imatanib), Guggulsterone, 4-hydroxy-3,6,7,8,3′,4′-hexamethoxyflavone, Hydroquinone, Ibuprofen, Indirubin-3′-oxime, Kaempferol, Monochloramine, Nafamostat mesilate, Obovatol, Oleandrin, Oleanolic acid, Panduratin A, Petrosaspongiolide M, Pinosylvin, Phytic acid, 20(S)-Protopanaxatriol, Rengyolone, Rottlerin, Saikosaponin-d, Sanguinarine, Sesaminol glucosides, Shikonins, Silymarin, Snake venom toxin (Vipera lebetina turanica), Spilanthol, Statins, Sulindac, 1,2,4-thiadiazolidine derivatives, Tomatidine, Vesnarinone, and Xanthoangelol D.

Other inhibitors of the activating phosphorylation of glutaminase C include binding molecules (e.g., antibodies and antigen-binding fragments thereof), siRNA and small molecule compounds. For example, binding molecules that specifically bind to glutaminase C at or near the site of the activating phosphorylation physically prevent the phosphorylation of glutaminase C by a kinase, such as mTOR. One example of a binding molecule is an antibody, or antigen-binding fragment thereof, that specifically binds to glutaminase C in a manner which sterically blocks the activating site from being phosphorylated. An anti-glutaminase C antibody that specifically binds to glutaminase C at the activating phosphorylation site prevents phosphorylation of glutaminase C by mTOR and is, therefore, an inhibitor of the activating phosphorylation. Also included in this embodiment are antibody fragments, T cell receptors and receptor fragments, dominant negative inhibitors, antisense, siRNA, and any other binding agents that block, inhibit, or down-regulate the phosphorylation of glutaminase C at or near the activating phosphorylation site.

In another embodiment, the activating phosphorylation of GAC is inhibited by a small molecule. Examples of small molecule inhibitors of activating phosphorylation or phosphorylation of glutaminase C include a compound selected from the group consisting of:

(i) a compound of formula (I):

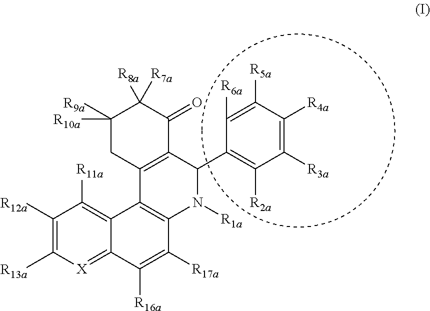

wherein:
the dotted circle identifies an active moiety;
X is independently —CR₁₄ₐ— or N;
R₁ₐ is independently H, OH, OR₁₄ₐ, C₁-C₆ alkyl, C₂-C₆ alkenyl, C₂-C₆ alkynyl, R₁₄ₐC(O)—, R₁₄ₐOC(O)—, R₁₄ₐS(O)—, or R₁₄ₐS(O)₂—,
R₂ₐ, R₃ₐ, R₄ₐ, R₅ₐ, and R₆ₐ are each independently H, halogen, NO₂, OH, OR₁₄ₐ, —SR₁₄ₐ, NH₂, NHR₁₄ₐ, NR₁₄ₐR₁₅ₐ, R₁₄ₐC(O)—, R₁₄ₐOC(O)—, R₁₄ₐC(O)O—, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; or
R₂ₐ and R₃ₐ, R₃ₐ and R₄ₐ, R₄ₐ and R₅ₐ, or R₅ₐ and R₆ₐ can combine to form a heterocyclic ring;
R₇ₐ, R₈ₐ, R₉ₐ, and R₁₀ₐ are each independently H, OH, NH₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, wherein the aryl, heteroaryl, and aryl C₁-C₆ alkyl are optionally substituted from 1 to 3 times with substitutents selected from the group consisting of halogen, OH, NH₂, C₁-C₆ alkyl, C₂-C₆ alkenyl, C₁-C₆ alkoxy, SH, and C₁-C₆ thioalkyl; and
R₁₁ₐ, R₁₂ₐ, R₁₃ₐ, R₁₄ₐ, R₁₅ₐ, R₁₆ₐ, and R₁₇ₐ are each independently H, halogen, OH, NO₂, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, each one of R₁₁ₐ-R₁₇ₐ optionally substituted with NH₂, OH, halogen, COOH, NO₂, and CN;

(ii) a compound of formula (II):

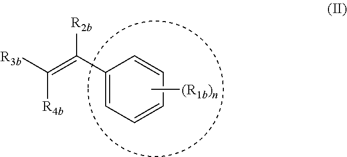

wherein:
the dotted circle identifies an active moiety;
n is an integer from 1 to 4;
R_{1b} is independently at each occurrence H, OH, OR_{5b}, halogen, CN, NO₂, NH₂, NHR_{5b}, NR_{5b}R_{6b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen;
R_{2b} is independently H, halogen, C₁-C₆ alkyl, C₂-C₆ alkenyl, C₂-C₆ alkynyl, C₃-C₆ cycloalkyl, C₄-C₇ cycloalkylalkyl, or mono or polycyclic aryl;
R_{3b} and R_{4b} are independently H, OR_{5b}, SR_{5b}, R_{5b}S(O)—, R_{5b}S(O)₂—, —COOR_{5b}, —C(O)NR_{5b}R_{6b}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; or
R_{3b} and R_{4b} can combine together to form a mono or polycyclic heterocyclyl or heteroaryl containing from 1-5 heteroatoms selected from the group consisting of nitrogen, sulfur, and oxygen, each formed heteroaryl or heterocyclyl optionally substituted with substituents selected from the group consisting of oxo, thio, amino, C₁-C₆ alkyl, C₂-C₆ alkenyl, and C₂-C₆ alkynyl; and
R_{5b} and R_{6b} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen, each one of R_{5b} or R_{6b} optionally substituted from 1 to 3 times with substituents selected from the group consisting of H, C₁-C₆ alkyl, C₂-C₆ alkenyl, C₂-C₆ alkynyl, C₃-C₆ cycloalkyl, and C₄-C₇ cycloalkylalkyl;

(iii) a compound of formula (III):

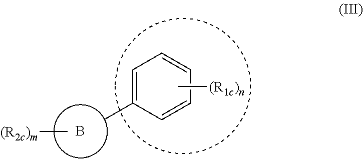

wherein:
the dotted circle identifies an active moiety;
m and n are integers from 1 to 4;
B is a substituted or unsubstituted mono or polycyclic aryl or mono or polycyclic heterocyclyl or heteroaryl with each cyclic unit containing from 1 to 5 heteroatoms selected from the group consisting of nitrogen, sulfur, and oxygen;
R_{1c} and R_{2c} are independently H, OH, OR_{3c}, halogen, CN, NO₂, COOH, NH₂, NHR_{3c}, NR_{3c}R_{4c}, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl with each cyclic unit containing from 1 to 5 heteroatoms selected from the group consisting of nitrogen, sulfur, and oxygen; and
R_{3c} and R_{4c} are independently H, C₁-C₆ alkyl, C₂-C₆ alkenyl, C₂-C₆ alkynyl, C₃-C₆ cycloalkyl, C₄-C₇ cycloalkylalkyl, aryl C₁-C₆ alkyl, mono or polycyclic aryl, or mono or polycyclic heteroaryl containing from 1 to 5 heteroatoms selected from the group consisting of nitrogen, sulfur, and oxygen; or

(iv) a compound comprising the active moiety of formula I, formula II, or formula III. Glutaminase C is then contacted with the compound under conditions effective to reduce the production of glutamate from glutamine in a cell or a tissue.

The inhibitor may further comprise an active moiety (linkable to other moieties), where the active moiety has the formula:

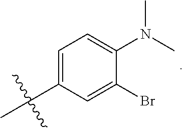 [SMILES: *c1ccc(N(C)C)c(Br)c1]

Exemplary compounds include any of the following:

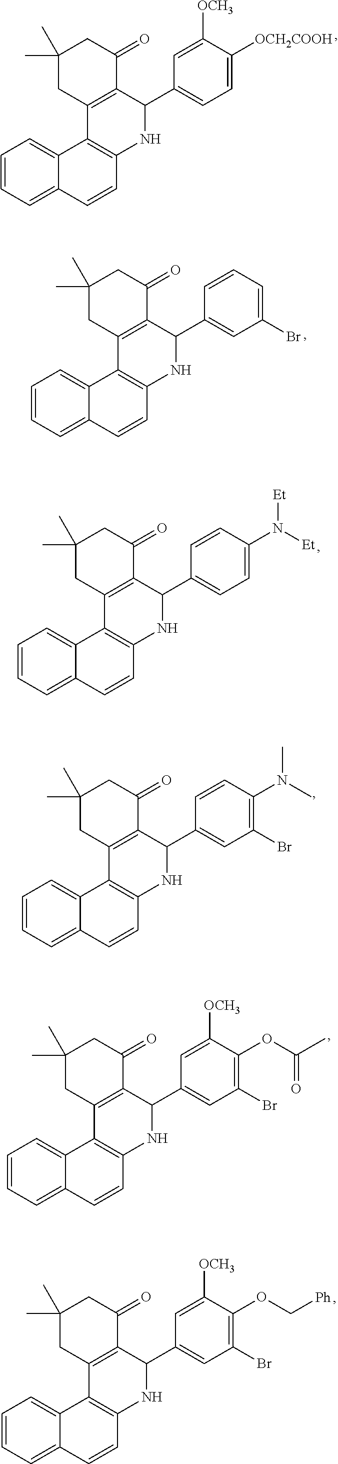

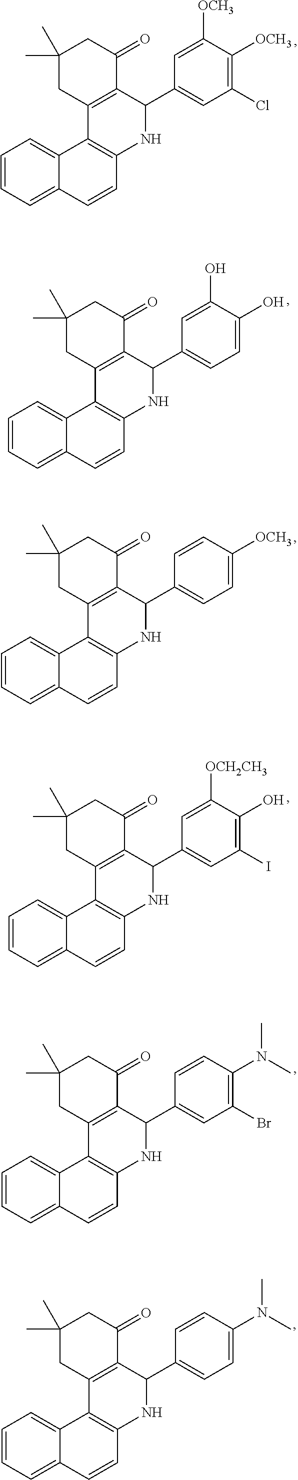 [SMILES: COc1cc(C2Nc3ccc4ccccc4c3C3=C2C(=O)CC(C)(C)C3)cc(Cl)c1OC ; CC1(C)CC(=O)C2=C(C1)c1c(ccc3ccccc13)NC2c1ccc(O)c(O)c1 ; COc1ccc(C2Nc3ccc4ccccc4c3C3=C2C(=O)CC(C)(C)C3)cc1 ; CCOc1cc(C2Nc3ccc4ccccc4c3C3=C2C(=O)CC(C)(C)C3)cc(I)c1O ; CN(C)c1ccc(C2Nc3ccc4ccccc4c3C3=C2C(=O)CC(C)(C)C3)cc1Br ; CN(C)c1ccc(C2Nc3ccc4ccccc4c3C3=C2C(=O)CC(C)(C)C3)cc1]

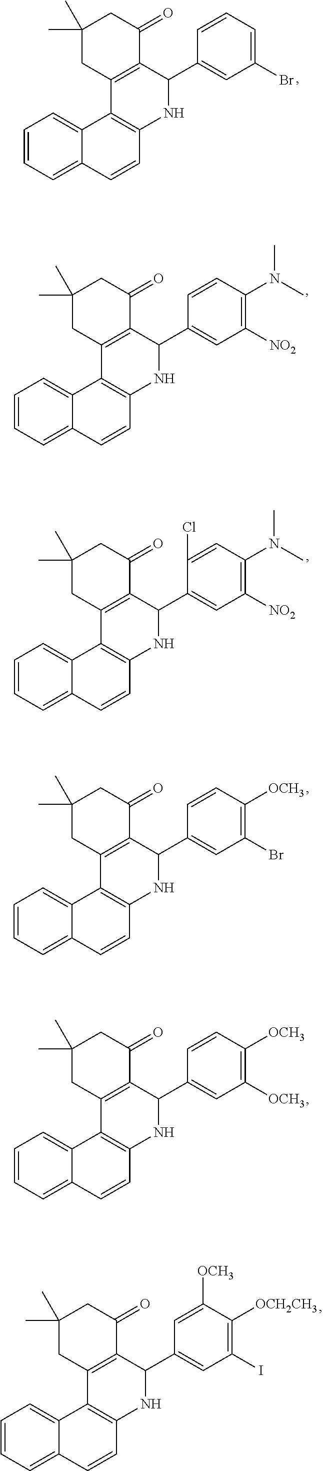 [SMILES: CC1(C)CC(=O)C2=C(C1)c1c(ccc3ccccc13)NC2c1cccc(Br)c1 ; CN(C)c1ccc(C2Nc3ccc4ccccc4c3C3=C2C(=O)CC(C)(C)C3)cc1[N+](=O)[O-] ; CN(C)c1cc(Cl)c(C2Nc3ccc4ccccc4c3C3=C2C(=O)CC(C)(C)C3)cc1[N+](=O)[O-] ; COc1ccc(C2Nc3ccc4ccccc4c3C3=C2C(=O)CC(C)(C)C3)cc1Br ; COc1ccc(C2Nc3ccc4ccccc4c3C3=C2C(=O)CC(C)(C)C3)cc1OC ; CCOc1c(I)cc(C2Nc3ccc4ccccc4c3C3=C2C(=O)CC(C)(C)C3)cc1OC]

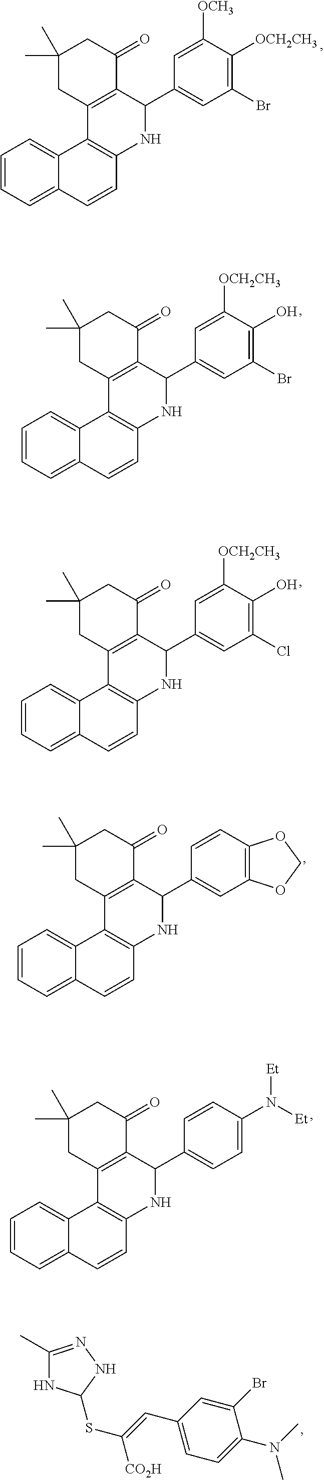

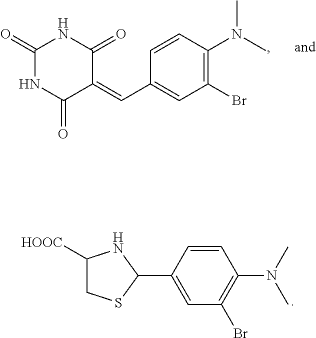 [SMILES: CN(C)c1ccc(C=C2C(=O)NC(=O)NC2=O)cc1Br ; CN(C)c1ccc(C2NC(C(=O)O)CS2)cc1Br]

Yet another example of an inhibitor is a compound of formula:

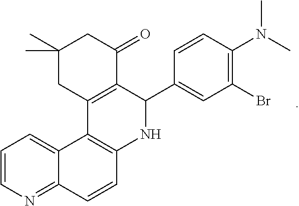 [SMILES: CN(C)c1ccc(C2Nc3ccc4ncccc4c3C3=C2C(=O)CC(C)(C)C3)cc1Br]

### Method of Treating or Preventing a Condition Mediated by the Activating Phosphorylation of Glutaminase C

Another aspect of the present invention relates to a method of treating or preventing a condition mediated by the activating phosphorylation of glutaminase C. The method involves selecting a subject having or at risk of having a condition mediated by the activating phosphorylation of glutaminase C and administering to said selected subject an inhibitor of the activating phosphorylation of glutaminase C. In particular, the activating phosphorylation occurs in the amino acid sequence of SEQ ID NO: 1. Any of the inhibitors of the activating phosphorylation of glutaminase C described above may be used in the methods described herein. For example, the inhibitor may be an inhibitor of a kinase that phosphorylates glutaminase C, an inhibitor of a transcription factor that regulates glutaminase C, a binding molecule (e.g., an anti-glutaminase C antibody) or a small molecule compound.

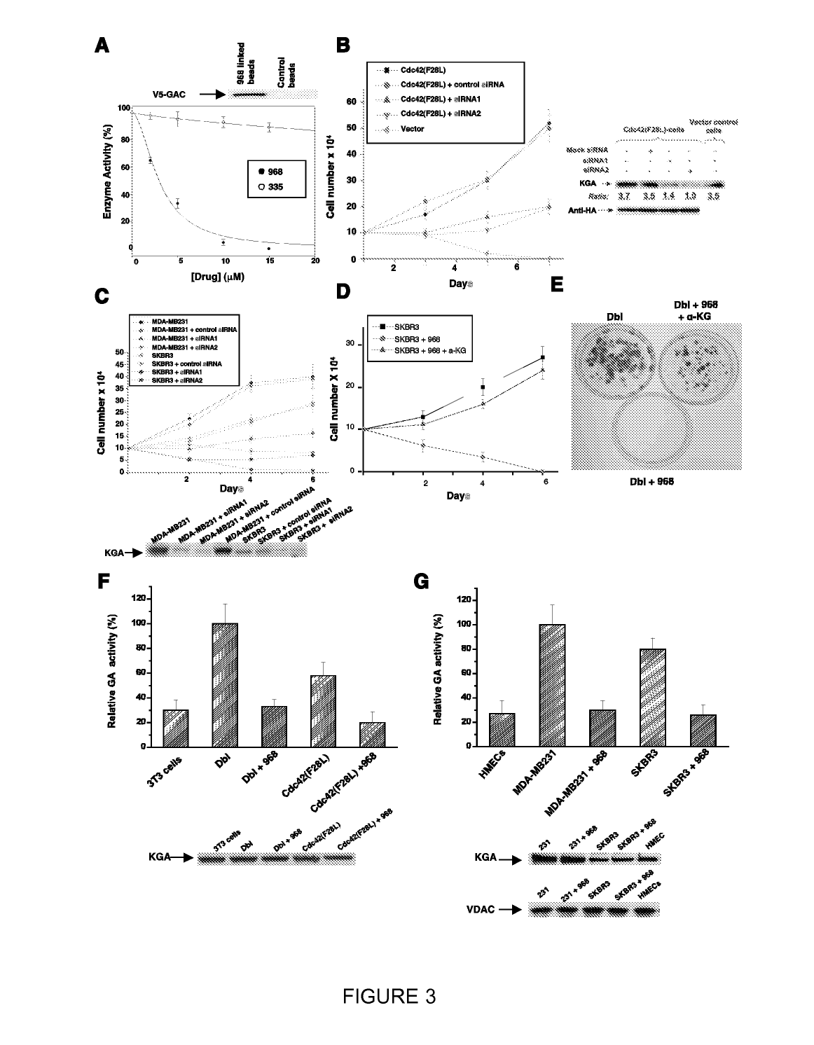

Although glutaminase C expression has been found to be increased in some cancers, Applicants have found that the participation of GAC is not limited to an increase in expression. Some cancer cells (such as the breast cancer cell line, SKBR3) have been found to exhibit GAC expression levels which are similar to normal cells, but are still dependent on the presence of GAC for cell growth (see FIG. 3C).

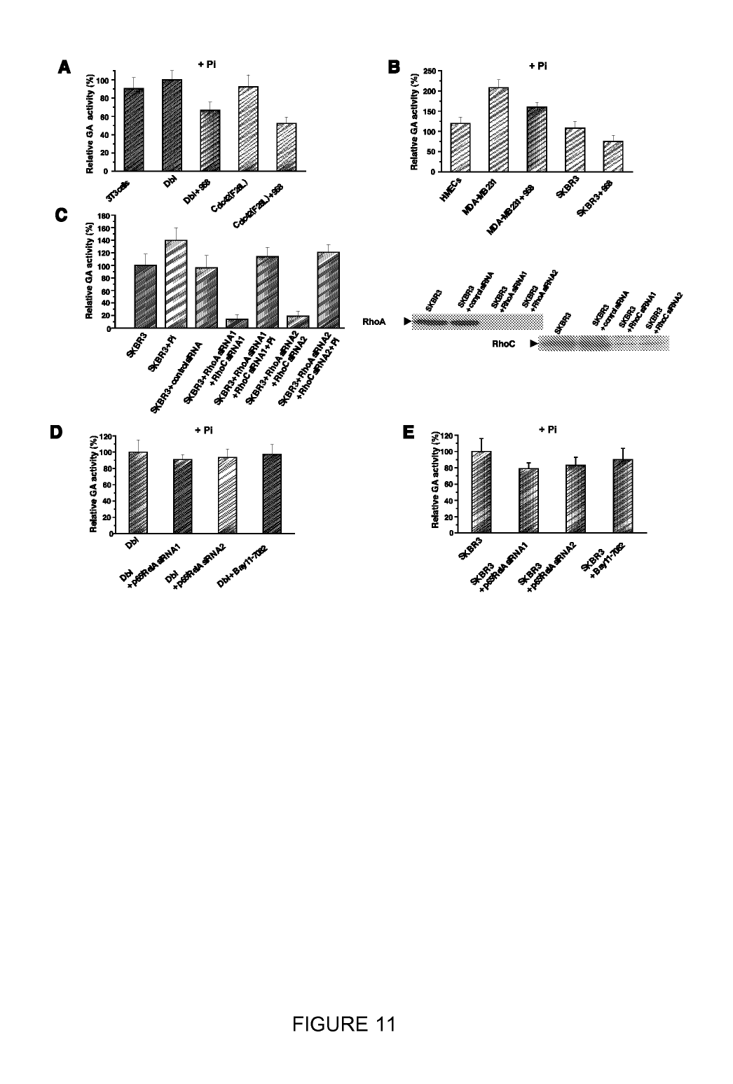

GAC isolated from cancer cells can show an elevated glutaminase activity level relative to GAC isolated from normal cells when assayed in the absence of phosphate, but in the presence of phosphate the enzymes isolated from both normal and cancer cells show a similar extent of activation per amount of GAC (FIG. 3G and FIG. 11B). Thus, the GAC in cancer cells is not dependent on the exogenous addition of phosphate to be active. Inhibition of phosphate-independent activation of GAC in cancer cells would inhibit the production of glutamate from glutamine.

One way in which the GAC activity from cancer cells may be increased relative to the GAC activity in normal cells is by an activating phosphorylation event that occurs on GAC. If the activation phosphorylation of GAC is inhibited, the ability for GAC to produce glutamate from glutamine is limited.

The activation state of GAC may vary among different cancer cells, regardless of the expression levels of GAC. A higher amount of activity may be referred to as “hyperactivity”. For example, Dbl transformed cells and Cdc42 F28L transformed cells contain similar levels of GAC as do untransformed NIH 3T3 cells. However, the GAC in the Dbl and Cdc42 transformed cells shows a higher activation than in the non-transformed cells, with the GAC from the Dbl cells being approximately twice as active than the GAC from the Cdc42 transformed cells (FIG. 3F). Thus, the GAC in the Dbl transformed cells is hyperactive. Inhibiting the hyperactivity of GAC in Dbl cells would limit the production of glutamate from glutamine by glutaminase C.

The conditions mediated by activating phosphorylation of GAC include, without limitation, breast cancer, lung cancer, brain cancer, pancreatic cancer, and colon cancer.

The subject receiving the inhibitor of the activating phosphorylation of GAC may be a human or a non-human animal (e.g. rat, mouse, pig, horse, monkey, cow, sheep, guinea pig, dog, and cat).

The inhibitors can be administered, e.g., by intravenous injection, intramuscular injection, subcutaneous injection, intraperitoneal injection, topical, sublingual, intraarticular (in the joints), intradermal, buccal, ophthalmic (including intraocular), intranasally (including using a cannula), or by other routes. The inhibitors of activating phosphorylation of GAC (e.g., formulae I, II, and/or III (as well as compounds comprising their active moieties)) can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. WO 97/11682, which is hereby incorporated by reference in its entirety) via a liposomal formulation (see, e.g., European Patent No. 736299, WO 99/59550, and WO 97/13500, which are hereby incorporated by reference in their entirety), via formulations described in WO 03/094886, which is hereby incorporated by reference in its entirety, or in some other form. The inhibitors can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al., Nature Reviews Drug Discovery 3:115 (2004), which is hereby incorporated by reference in its entirety). The inhibitors can be administered locally, for example, at the site of injury to an injured blood vessel. The inhibitors can be coated on a stent. The inhibitors can be administered using high-velocity transdermal particle injection techniques using the hydrogel particle formulation described in U.S. Patent Publication No. 20020061336, which is hereby incorporated by reference in its entirety. Additional particle formulations are described in WO 00/45792, WO 00/53160, and WO 02/19989, which are hereby incorporated by reference in their entirety. An example of a transdermal formulation containing plaster and the absorption promoter dimethylisosorbide can be found in WO 89/04179, which is hereby incorporated by reference in its entirety. WO 96/11705, which is hereby incorporated by reference in its entirety, provides formulations suitable for transdermal administration.

### Detection, Diagnosis and Imaging

As noted above, GAC is overexpressed or “hyperactive” in proliferating cancer cells as compared normal cells. Accordingly, in certain embodiments, the present invention includes methods of detecting and imaging tumor cells by contacting tumors with an agent that specifically binds to active GAC, e.g., an agent that binds to active GAC coupled to a detectable label and/or a therapeutic agent. These methods may generally be applied to a variety of tumors, including those specifically described herein.

Diagnostic or detection methods of the present invention generally involve contacting a biological sample with a reagent, such as an antibody or fragment thereof described herein that specifically binds to active GAC, under conditions that allow binding and determining whether the reagent preferentially binds to the sample as compared to a control biological sample or predetermined cut-off value, thereby indicating the presence of active GAC in the sample. In various embodiments, the biological sample is, e.g., blood, serum, saliva, urine, sputum, a cell swab sample, or a tissue biopsy. The biological sample may be obtained from an animal, such as a human. In certain embodiments, the predetermined cut-off value is the amount detected in a normal control biological sample. In other embodiments, the predetermined cut-off value is 1.5 or 2 times the amount detected in a normal control individual or biological sample.

Bound reagent may be detected using procedures described herein and known in the art. In certain embodiments, methods of the present invention are practiced using reagents that are conjugated to a detectable label, e.g., a fluorophore, to facilitate detection of bound reagent. In addition, antibodies can be detected with anti-constant region secondary agents and may be useful for immunochemistry assays of tissues to assess tissue distribution and expression of the TCblR. These include, for example, RIA, ELISA, precipitation, agglutination, complement fixation and immuno-fluorescence.

An enzyme label can be detected by any of the currently utilized colorimetric, spectrophotometric, fluorospectro-photometric or gasometric techniques. Many enzymes used in these procedures are known and can be utilized. Examples are peroxidase, alkaline phosphatase, β-glucuronidase, β-D-glucosidase, β-D-galactosidase, urease, glucose oxidase plus peroxidase, galactose oxidase plus peroxidase and acid phosphatase.

Assessing tissue distribution and amount of active GAC using, e.g., immunochemistry assays and diagnostic imaging techniques, can be used to detect and diagnose a tumor, cancer or hyperproliferative disease or disorder in a subject. In particular, an increased amount of active GAC relative to the surrounding tissue is indicative of increased cellular proliferation. The relative expression of active GAC can be monitored over time to monitor the state or course of a disease, e.g., progression or regression.

In the context of tumor detection in a subject, a detection reagent, such as an antibody or fragment thereof, is typically coupled to a detectable label and delivered to a subject. The subject is then examined and the presence and/or location of detectable label determined and correlated with the presence of a tumor. Typically, the presence of a tumor is associated with the detection of at least two-fold, at least three-fold, or at least five-fold as much label as detected in a normal control patient.

For example, the presence of tumor cells in a tissue sample obtained from a patient is determined by comparing the amount of binding of an anti-active GAC antibody or fragment thereof to the tissue sample to the amount of binding to a control normal tissue sample or a predetermined cut-off value. Typically, the presence of tumor cells is associated with at least two-fold, at least three-fold, or at least five-fold as much bound active GAC binding agent as detected in a normal control tissue sample.

These methods may be readily adapted for prognostic and monitoring purposes. For example, the amount of active GAC detected using an antibody or fragment thereof may be determined before and after treatment. If the amount is reduced following treatment, it suggests that the treatment is efficacious. However, if the amount is increased following treatment, it suggests that the treatment is not efficacious. Similarly, the location of active GAC detected using a reagent, such as an antibody or fragment thereof, may be determined at first and second time points. If active GAC is detected at different or new locations in the body at the second, later time point as compared to the first, earlier time point, it indicates that the tumor is growing or has metastasized.

The invention contemplates the use of any type of detectable label, including, e.g., visually detectable labels, such as, e.g., dyes, fluorophores, and radioactive labels. In addition, the invention contemplates the use of magnetic beads and electron dense substances, such as metals, e.g., gold, as labels. A wide variety of radioactive isotopes may be used, including, e.g., ¹⁴C, ³H, ^{99m}Tc, ¹²³I, ¹³¹I, ³²P, ¹⁹²Ir¹⁰³Pd, ¹⁹⁸Au, ¹¹¹In, ⁶⁷Ga, ²⁰¹TI, ¹⁵³Sm, ¹⁸F an ⁹⁰Sr. Other radioisotopes that may be used include, e.g., thallium-201 or technetium 99m. In certain embodiments, the detectable label is a CT contrast agent, also referred to as “dyes.” Examples of commonly used contrast agents include iodine, barium, barium sulfate, and gastrografin. In other embodiments, the detectable agent is a fluorophore, such as, e.g., fluorescein or rhodamine. A variety of biologically compatible fluorophores are commercially available.

Accordingly, the present invention includes a method of detecting or diagnosing a condition mediated by activating phosphorylation of GAC (e.g., tumor, cancer, or hyperproliferative disease or disorder) in a subject, comprising contacting a biological sample from the subject with a reagent that specifically binds to the activating phosphorylation site within SEQ ID NO:1 of GAC, and identifying the phosphorylated GAC-reagent conjugate or complex, thereby detecting a condition mediated by the activating phosphorylation of GAC. In certain embodiments, the reagent recognizes GAC that has been phosphorylated at the activating phosphorylation site. In another embodiment, the reagent recognizes a mutant GAC that is in a constitutively active conformation, such as S103A.

An isolated antibody, or fragment thereof, can be lyophilized for storage or formulated into various solutions known in the art for solubility and stability and consistent with safe administration into animals, including humans. An antibody composition may contain antibodies of multiple isotypes or antibodies of a single isotype. An antibody composition may contain unmodified antibodies, or the antibodies may have been modified in some way, e.g., chemically or enzymatically. Thus an antibody composition may contain intact Ig molecules or fragments thereof, i.e., Fab, F(ab′)₂, or Fc domains. In particular embodiments, antibodies may be in solution or attached to a surface such as a polystyrene or latex plate or bead. In certain embodiments, antibodies may be conjugated to an agent as described herein.

The antibodies and fragments described herein also may be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980), which is hereby incorporated by reference in its entirety.

The antibodies and fragments disclosed herein may also be formulated as immunoliposomes. A “liposome” is a small vesicle composed of various types of lipids, phospholipids and/or surfactant that is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA, 82:3688 (1985); Hwang et al., Proc. Natl. Acad. Sci. USA, 77:4030 (1980); U.S. Pat. Nos. 4,485,045 and 4,544,545; and PCT Publication No. WO 97/38731 published Oct. 23, 1997, which are hereby incorporated by reference in their entirety. Liposomes with enhanced circulation time are disclosed in U.S. Pat. No. 5,013,556.

### Screening Methods

Another aspect of the present invention relates to a method of screening for compounds capable of treating or preventing cancer. The method involves providing a cell or tissue under conditions effective for the cell or tissue to produce glutamate from glutamine as a result of glutaminase C activity. A plurality of candidate compounds is provided to contact the cell or tissue and the candidate compounds which inhibit the activating phosphorylation of glutaminase C as a result of said contacting are identified. In particular, the compounds inhibit activating phosphorylation of GAC within the amino acid sequence of SEQ ID NO: 1.

In certain embodiments, the compounds are identified by screening candidates for their ability to inhibit the activating phosphorylation in vitro or in vivo. Any assay suitable for determining the inhibition of phosphorylation may be used, and a variety of such assays are known and available in the art.

Candidate compounds may be screened individually, e.g., when a specific molecule is predicted to function as an inhibitor. Alternatively, a plurality of compounds may be screened.

In certain embodiments, the method of screening for compounds capable of treating or preventing cancer include providing a cancer cell or cancer tissue under conditions effective to produce glutamate from glutamine as a result of glutaminase C activity, providing a plurality of candidate compounds, contacting the cancer cell or cancer tissue with the candidate compounds under conditions effective for the activating phosphorylation, and identifying the candidate compounds which inhibit the activating phosphorylation of GAC within the amino acid sequence of SEQ ID NO: 1. In a related embodiment, the cancer cell or cancer tissue is lysed after contacting the candidate compounds, and the identifying step uses an antibody that specifically binds phosphorylated or active GAC.

In certain embodiments, the amino acid residue X of SEQ ID NO: 1 is valine or alanine. In certain embodiments, the activating phosphorylation occurs at serine 95 of the amino acid sequence of SEQ ID NO: 2 or at serine 103 of SEQ ID NO: 3. In certain embodiments, the cancer cell or cancer tissue is cultured in the presence of radiolabelled phosphate, and the activating phosphorylation of GAC is determined by detecting radiolabelled GAC.

### Pharmaceutical Compositions and Kits

Pharmaceutical compositions can comprise an inhibitor of activating phosphorylation of GAC and a pharmaceutically acceptable carrier and, optionally, one or more additional active agent(s) as discussed below.

The amount of active ingredient that may be combined with the carrier materials to produce a single dosage form will vary depending upon the host treated and the particular mode of administration. For example, a formulation intended for the oral administration of humans may vary from about 5% to about 95% of the total composition. Dosage unit forms will generally contain between from about 1 mg to about 500 mg of active ingredient.

Any pharmaceutically acceptable liquid carrier suitable for preparing solutions, suspensions, emulsions, syrups and elixirs may be employed in the composition of the invention Inhibitors may be dissolved or suspended in a pharmaceutically acceptable liquid carrier such as water, an organic solvent, or a pharmaceutically acceptable oil or fat, or a mixture thereof. The liquid composition may contain other suitable pharmaceutical additives such as solubilizers, emulsifiers, buffers, preservatives, sweeteners, flavoring agents, suspending agents, thickening agents, coloring agents, viscosity regulators, stabilizers, osmo-regulators, or the like. Examples of liquid carriers suitable for oral and parenteral administration include water (particularly containing additives as above, e.g., cellulose derivatives, preferably sodium carboxymethyl cellulose solution), alcohols (including monohydric alcohols and polyhydric alcohols, e.g., glycols) or their derivatives, or oils (e.g., fractionated coconut oil and arachis oil). For parenteral administration the carrier may also be an oily ester such as ethyl oleate or isopropyl myristate.

Pharmaceutically acceptable salts include, but are not limited to, amine salts, such as but not limited to, N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine, and tris (hydroxymethyl) aminomethane; alkali metal salts, such as but not limited to, lithium, potassium, and sodium; alkali earth metal salts, such as but not limited to, barium, calcium, and magnesium; transition metal salts, such as but not limited to, zinc; and other metal salts, such as but not limited to, sodium hydrogen phosphate and disodium phosphate; and also including, but not limited to, salts of mineral acids, such as but not limited to, hydrochlorides and sulfates; and salts of organic acids, such as but not limited to, acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, cycloalkyl and heterocyclyl esters of acidic groups, including, but not limited to, carboxylic acids, phosphoric acids, phosphinic acids, sulfonic acids, sulfinic acids, and boronic acids. Pharmaceutical acceptable enol ethers include, but are not limited to, derivatives of formula C═C(OR) where R is hydrogen, alkyl, alkenyl, alkynyl, aryl, heteroaryl, cycloalkyl, or heterocyclyl. Pharmaceutically acceptable enol esters include, but are not limited to, derivatives of formula C═C(OC(O)R) where R is hydrogen, alkyl, alkenyl, alkynyl, aryl, heteroaryl, cycloalkyl, or heterocyclyl. Pharmaceutical acceptable solvates and hydrates are complexes of a compound with one or more solvent or water molecules, or 1 to about 100, or 1 to about 10, or one to about 2, 3, or 4, solvent or water molecules.

It will be understood, however, that the specific dose level for any particular patient will depend upon a variety of factors, including the activity of the specific inhibitor employed, the age, body weight, general health, sex, diet time of administration, route of administration, rate of excretion, drug combination and the severity of the particular disease undergoing therapy.

All methods comprise administering to the subject in need of such treatment an effective amount of one or more inhibitors of the present invention.

A subject or patient in whom administration of the therapeutic inhibitor is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The inhibitors of the present invention can be administered alone or as an active ingredient of a formulation. The inhibitors of the present invention can be administered in a form where the active ingredient is substantially pure.

Numerous standard references are available that describe procedures for preparing various formulations suitable for administering the inhibitors according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition), which are hereby incorporated by reference in their entirety.

In certain embodiments, the amount of inhibitor provided and administered is sufficient to result in tumor regression, as indicated by a statistically significant decrease in the amount of viable tumor, for example, at least a 50% decrease in tumor mass, or by altered (e.g., decreased with statistical significance) scan dimensions. In other embodiments, the amount administered is sufficient to inhibit cell growth or proliferation.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described inhibitor of activating phosphorylation of GAC in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of an antibody of the present disclosure, for treatment of a disease or condition of interest in accordance with teachings herein.

A pharmaceutical composition may be in the form of a solid or liquid. In one embodiment, the carrier(s) are particulate, so that the compositions are, for example, in tablet or powder form. The carrier(s) may be liquid, with the compositions being, for example, oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition intended for either parenteral or oral administration should contain an amount of an inhibitor of activating phosphorylation of GAC as herein disclosed such that a suitable dosage will be obtained. Typically, this amount is at least 0.01% of the inhibitor in the composition. When intended for oral administration, this amount may be varied to be between 0.1 and about 70% of the weight of the composition. Certain oral pharmaceutical compositions contain between about 4% and about 75% of the inhibitor. In certain embodiments, pharmaceutical compositions and preparations according to the present invention are prepared so that a parenteral dosage unit contains between 0.01 to 10% by weight of the inhibitor prior to dilution.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The pharmaceutical composition in solid or liquid form may include an agent that binds to the inhibitor and thereby assists in the delivery of the compound. Suitable agents that may act in this capacity include other monoclonal or polyclonal antibodies, one or more proteins or a liposome. The pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The pharmaceutical compositions may be prepared by methodology well known in the pharmaceutical art. For example, a pharmaceutical composition intended to be administered by injection can be prepared by combining a composition that comprises an inhibitor of the activating phosphorylation of GAC as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the inhibitor composition so as to facilitate dissolution or homogeneous suspension of the inhibitor in the aqueous delivery system.

The compositions may be administered in a therapeutically effective amount, which will vary depending upon a variety of factors including the activity of the specific inhibitor employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

Compositions comprising inhibitors of the activating phosphorylation of GAC may also be administered simultaneously with, prior to, or after administration of one or more other therapeutic agents. Such combination therapy may include administration of a single pharmaceutical dosage formulation which contains a compound of the invention and one or more additional active agents, as well as administration of compositions comprising inhibitors and each active agent in its own separate pharmaceutical dosage formulation. For example, an inhibitor as described herein and the other active agent can be administered to the patient together in a single oral dosage composition such as a tablet or capsule, or each agent administered in separate oral dosage formulations. Similarly, an inhibitor as described herein and the other active agent can be administered to the patient together in a single parenteral dosage composition such as in a saline solution or other physiologically acceptable solution, or each agent administered in separate parenteral dosage formulations. Where separate dosage formulations are used, the compositions comprising inhibitors and one or more additional active agents can be administered at essentially the same time, i.e., concurrently, or at separately staggered times, i.e., sequentially and in any order; combination therapy is understood to include all these regimens.

Thus, in certain embodiments, also contemplated is the administration of inhibitor compositions of this disclosure in combination with one or more other therapeutic agents. Such therapeutic agents may be accepted in the art as a standard treatment for a particular disease state as described herein, such as a tumor, cancer or a proliferative disease or disorder. Exemplary therapeutic agents contemplated include cytokines, steroids, chemotherapeutics, radiotherapeutics, or other active and ancillary agents.

The present invention also includes kits useful in performing assays using the inhibitors of activating phosphorylation of GAC. These kits include a suitable container comprising one or more inhibitors of activating phosphorylation or one or more detection reagents described herein. The detection reagent may be conjugated or unconjugated to a detectable label. In addition, if the detection reagent is supplied in a labeled form suitable for an indirect binding assay, the kit may further comprise reagents useful in performing the appropriate indirect assay. For example, the kit may include one or more suitable containers comprising enzyme substrates or derivatizing agents, depending on the nature of the label. Control samples and/or instructions may also be included.

The invention further provides kits for detecting active GAC or cells or tissues expressing active GAC in a sample, wherein the kits contain at least one antibody, polypeptide, polynucleotide, or vector as described herein. In certain embodiments, a kit may comprise buffers, enzymes, labels, substrates, beads or other surfaces to which the antibodies of the invention are attached, and the like, and instructions for use. In particular embodiments, a kit comprises a composition comprising a physiologically acceptable carrier and a therapeutically effective amount of an active GAC detection reagent described herein.

### EXAMPLES

The Examples set forth below are for illustrative purposes only and are not intended to limit, in any way, the scope of the present invention.

### Example 1

### Identification of Glutaminase as the Target of 968

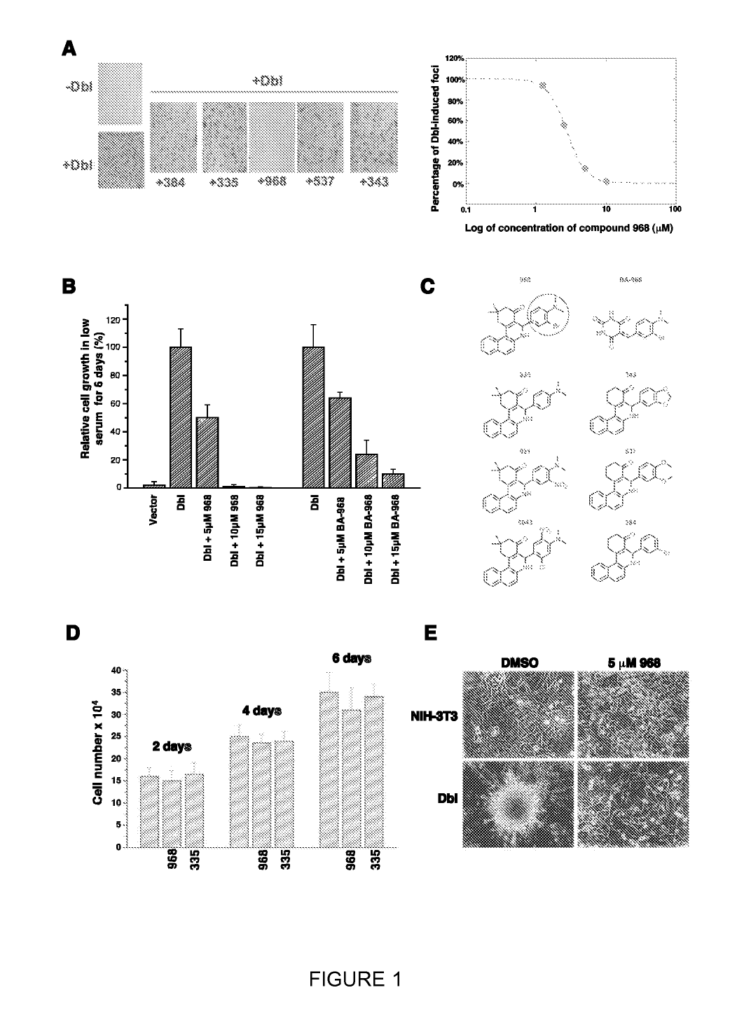

Compound 968 (5-[3-bromo-4-(dimethylamino)phenyl]-2,2-dimethyl-2,3,5,6-tetrahydrobenzo[a]) was obtained from SPECS (Netherlands; CAS registry #311795-38-7). In order to identify the molecular target of 968, its active moiety (3-bromo-4-(dimethylamino)benzyl) (See FIG. 1C) (ChemBridge Corporation, San Diego; CAS registry #56479-63-1) was incorporated into biotin hydrazide by reacting 3-bromo-4-(dimethylamino)benzaldehyde or formaldehyde (as a negative control) at a 5-fold molar excess overnight at 42° C., followed by reduction with cyanoborohydride coupling buffer. The 968-biotin adduct (MW=554 Da) was confirmed by mass spectrometry and incubated with streptavidin-agarose beads equilibrated with cell lysis buffer (5 mM MgCl₂, 120 mM NaCl, 10 mM HEPES, pH 7.4, 0.5% NP-40, 10 μg/ml leupeptin, and 10 μg/ml aprotinin) prior to incubation with lysates from NIH 3T3 cells stably expressing Cdc42(F28L) (5 ml containing ˜2 mg/ml total protein) for 2 h at 4° C. The beads were washed 3× with cold lysis buffer and pelleted by centrifugation and the associated proteins were resolved by SDS-PAGE. A silver-stained protein band (Mᵣ ˜66 kDa) that bound specifically to the 968-biotin beads and not to the control beads was excised and analyzed by mass spectrometry at the Harvard Microchemistry Facility (Cambridge, Mass.) and identified as mouse KGA isoform-2 (accession number NP_{—}001106854), the mouse ortholog of the human GAC isoform.

### Example 2

### Mitochondrial Preparations

Mitochondrial preparations were obtained using the mitochondria isolation kit from QIAGEN (Cat #37612). A suspension containing 2×10⁷ cells was transferred into a 50 ml conical tube and centrifuged at 500×g for 10 minutes at 4° C. The pellets were resuspended in 2 ml of ice-cold lysis buffer (supplied by QIAGEN) and incubated for 10 minutes at 4° C. using an end-over-end shaker. The lysates were centrifuged at 1000×g for 10 minutes at 4° C., and the pellets were resuspended in a buffer supplied by the manufacturer and disrupted completely by using a blunt-ended, 23-gauge needle and a syringe, followed by centrifugation at 6000×g for 20 minutes at 4° C. The pellets were resuspended in 100 μl of 20 mM Hepes, pH 7.4, 150 mM NaCl, 1% NP-40, 20 mM β-glycerolphosphate, 1 mM sodium orthovanadate, and 20 mM sodium fluoride and assayed for GA activity as previously described (Kenny et al., “Bacterial Expression, Purification and Characterization of Rat Kidney-Type Mitochondrial Glutaminase,” Protein Expr. Purif. 31:140-148 (2003), which is hereby incorporated by reference in its entirety) and further outlined below for assaying recombinant enzyme, except that the recombinant protein was replaced by 20 μl of resuspended mitochondrial lysate.

### Example 3

### RNAi

All knock-downs were performed by using Stealth Select RNAi Duplexes from Invitrogen that were transiently transfected into cells using Lipofectamine 2000. A non-specific oligonucleotide was used as a negative control. The relative knock-down efficiencies were determined using the following antibodies: A polyclonal antibody that recognizes both isoforms of KGA, an anti-RhoC polyclonal antibody from Santa Cruz, an anti-RhoA monoclonal antibody, and an anti-p65/RelA polyclonal antibody from Cell Signaling.

### Example 4

### Assays of Recombinant Glutaminase Activity

Glutaminase activity assays were performed on recombinant enzyme as previously described (Kenny et al., “Bacterial Expression, Purification and Characterization of Rat Kidney-Type Mitochondrial Glutaminase,” Protein Expr. Purif. 31:140-148 (2003), which is hereby incorporated by reference in its entirety). A plasmid encoding mouse GAC (residues 128-603) was cloned into the pET28a vector and the protein was expressed with an N-terminal histidine (His)-tag. The tag was cleaved using thrombin and the protein was further purified by anion-exchange and gel-filtration chromatography. Recombinant GAC (1 μM) was incubated with varying concentrations of 968 in 57 mM Tris-Acetate (pH 8.6) and 0.225 mM EDTA by rotating at 37° C. for 30 minutes, in a final volume of 80 μl. Compound 968 was diluted in DMSO such that the volume added was constant (5 μl) for all samples, ensuring that the concentration of DMSO (6.3% v/v) was the same in each of the assay incubations. A glutamine solution was then added to give a final volume of 115 μl and a final concentration of 17 μM. The reaction proceeded at 37° C. for 1 h and was stopped by adding 10 μl of ice-cold 3M HCl. An aliquot of the quenched reaction mixture (10 μl) was added to an incubation containing 114 mM Tris-HCl (pH 9.4), 0.35 mM ADP, 1.7 mM NAD and 6.3 U/ml glutamate dehydrogenase to give a final volume of 228 μl. The reaction mixture was incubated at room temperature for 45 minutes and the absorbance at 340 nm was recorded for each sample against a water blank. The absorbance of the sample with just the cocktail mixture was subtracted from each reading to calculate the activity of the enzyme.

### Example 5

### Glutaminase C Expression

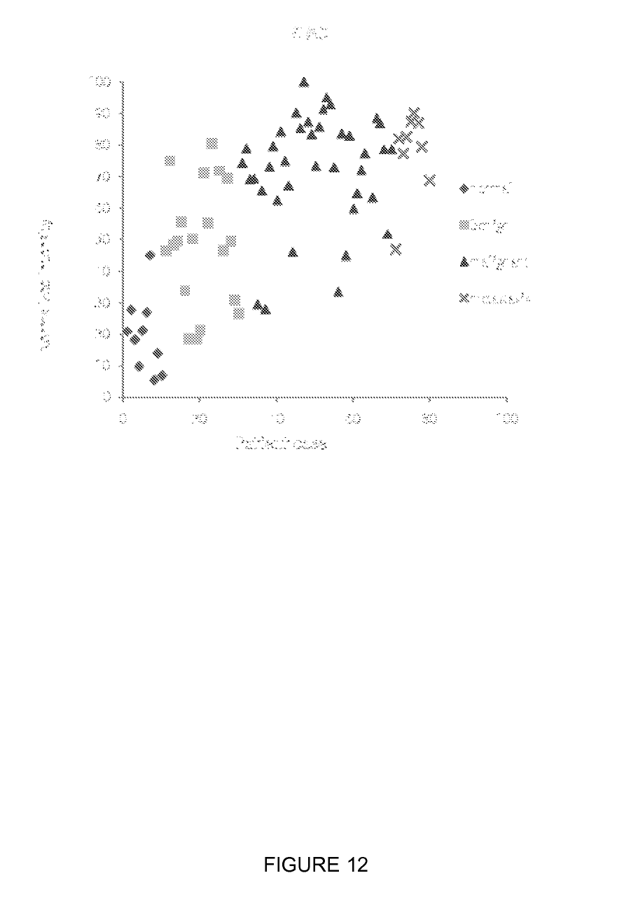

Serial slides of a breast tissue array were obtained from Biomax U.S.A. and were probed with either an antibody against GAC, or an antibody against actin (control). The expression of GAC was then normalized to the expression of actin for each sample. An increase in GAC protein levels were observed in transformed breast tissues. See FIG. 12.

### Example 6

### Comparison of Glutaminase C and Kidney-Glutaminase Expression

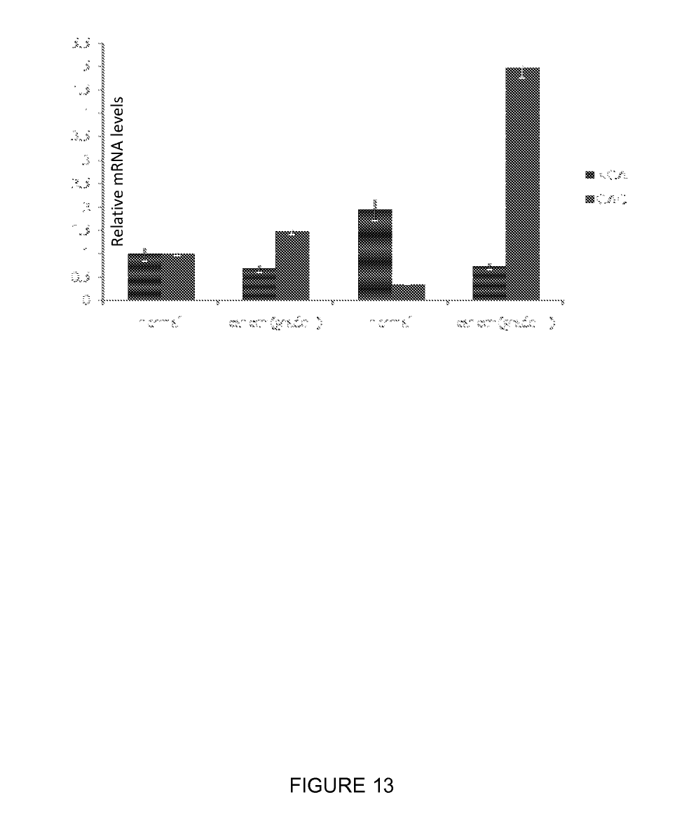

Total RNA was extracted from normal or cancerous breast tissues, and complementary DNA was then synthesized. Quantitative PCR (qPCR) was performed in triplicate using primer sets to amplify KGA, GAC or GAPDH, the normalizer/housekeeping gene, on an ABI7500 Fast Real-Time PCR System. Relative quantification studies were performed with the ABI7500 Fast System Sequence Detection Software. See FIG. 13.

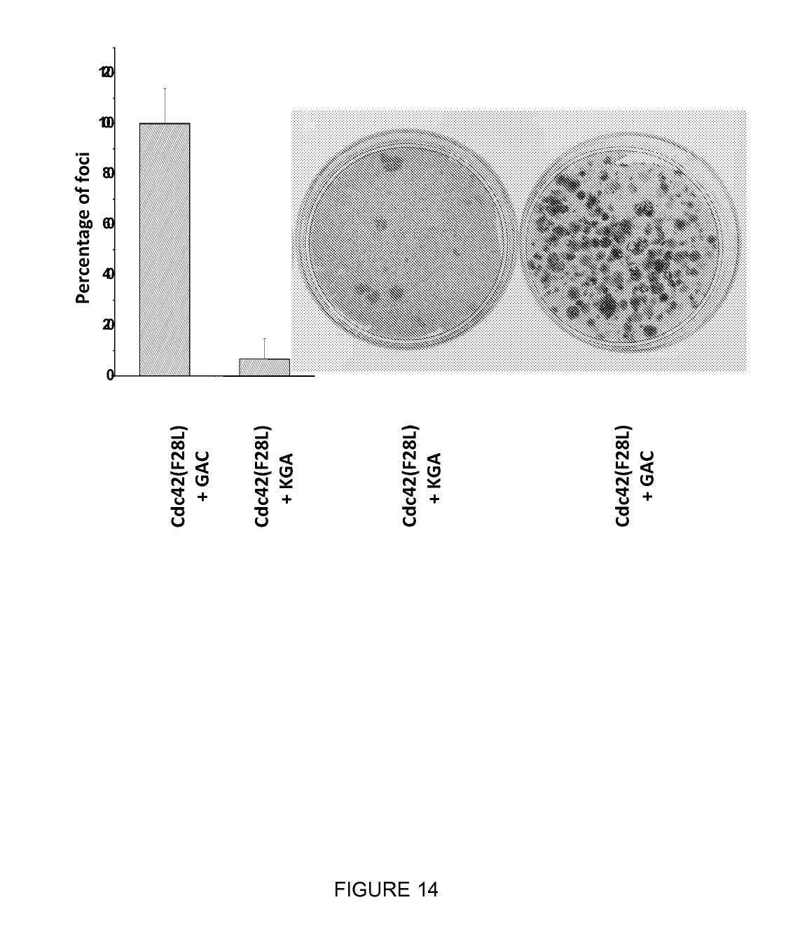

NIH 3T3 cells expressing a constitutively active form of Cdc42, Cdc42 (F28L), were transiently transfected with either DNA encoding GAC or KGA. The cells were then allowed to grow under conditions permissive for focus formation and the number of foci were then counted and scored. See FIG. 14.

### Example 7

### Glutaminase C Phosphorylation

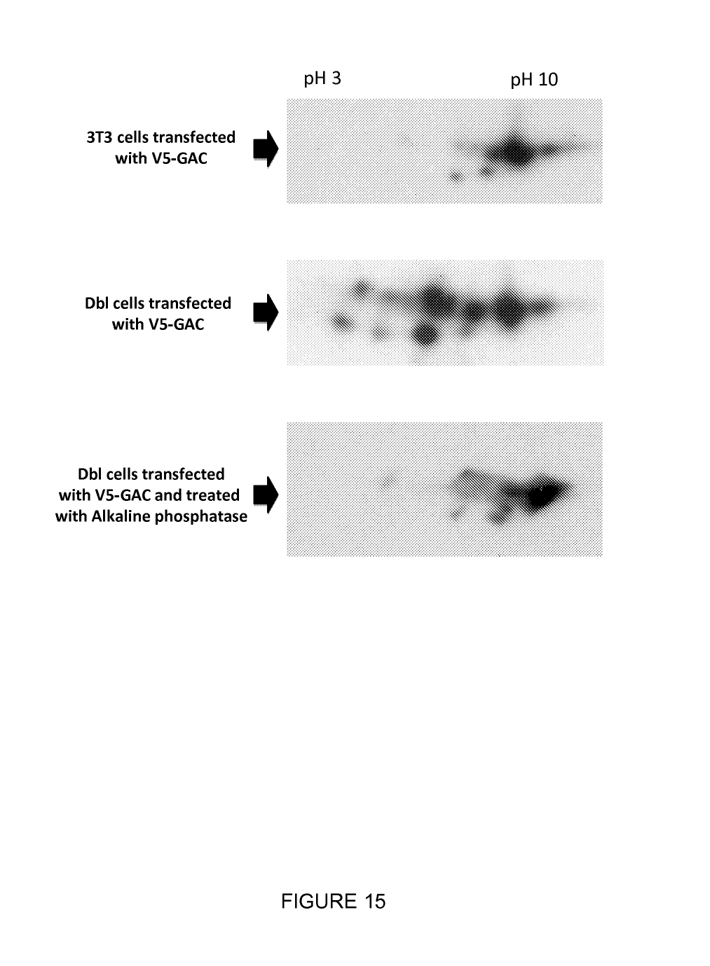

NIH 3T3 cells or NIH 3T3 cells stably expressing the Dbl oncogene, were transiently transfected with DNA encoding a V5-tagged GAC. The cells were then harvested, and the ectopically expressed GAC was isolated by immunoprecipitation via the V5 tag. The V5-GAC obtained from one of the Dbl samples was additionally treated with alkaline phosphatase under dephosphorylation conditions. The samples were then subjected to 2-D gel analysis to separate the V5-GAC by charge and size, and the V5-tagged GAC was visualized by Western blotting using an anti-V5 antibody. Multiple modification states of GAC were detected on GAC isolated from NIH 3T3 cells expressing Dbl as compared to the GAC isolated from untransformed NIH 3T3 cells. The multiple modification states of GAC were reversed when the protein was treated with alkaline phosphatase, suggesting that the modifications are phosphorylations. See FIG. 15.

### Example 8

### Relationship of Glutaminase C Phosphorylation to Basal Glutaminase Activity

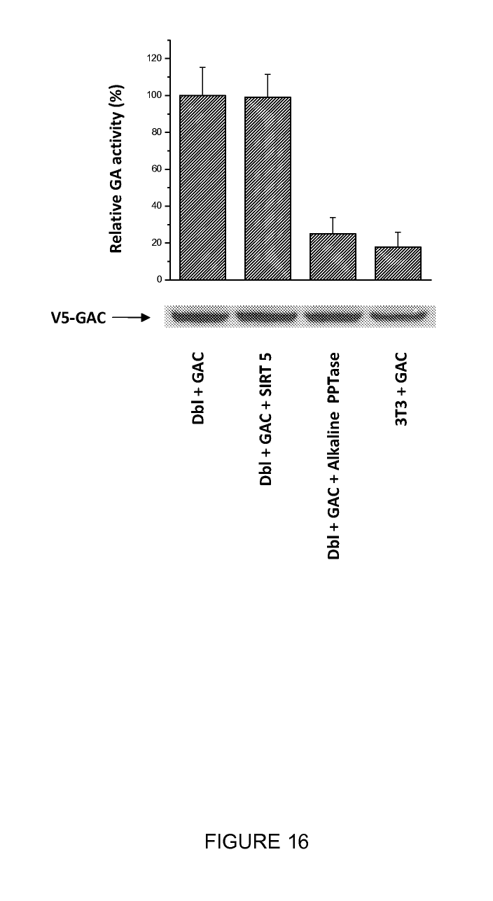

NIH 3T3 cells or NIH 3T3 cells stably expressing the Dbl oncogene, were transiently transfected with DNA encoding a VS-tagged GAC. The cells were then harvested and the ectopically expressed GAC was isolated by immunoprecipitation via the V5 tag. The V5-GAC obtained from one of the Dbl samples was additionally treated with alkaline phosphatase under dephosphorylation conditions. See FIG. 16. The samples were then assayed for glutaminase activity in the absence of phosphate (top panel) and the relative expression levels of V5-GAC was determined by Western blotting using an anti-V5 antibody (bottom panel). The dephosphorylation of GAC isolated from Dbl cells resulted in a 75% reduction of basal glutaminase (phosphate independent) activity.

### Example 9

### Inhibition of Glutaminase C Phosphorylation by Compound 968

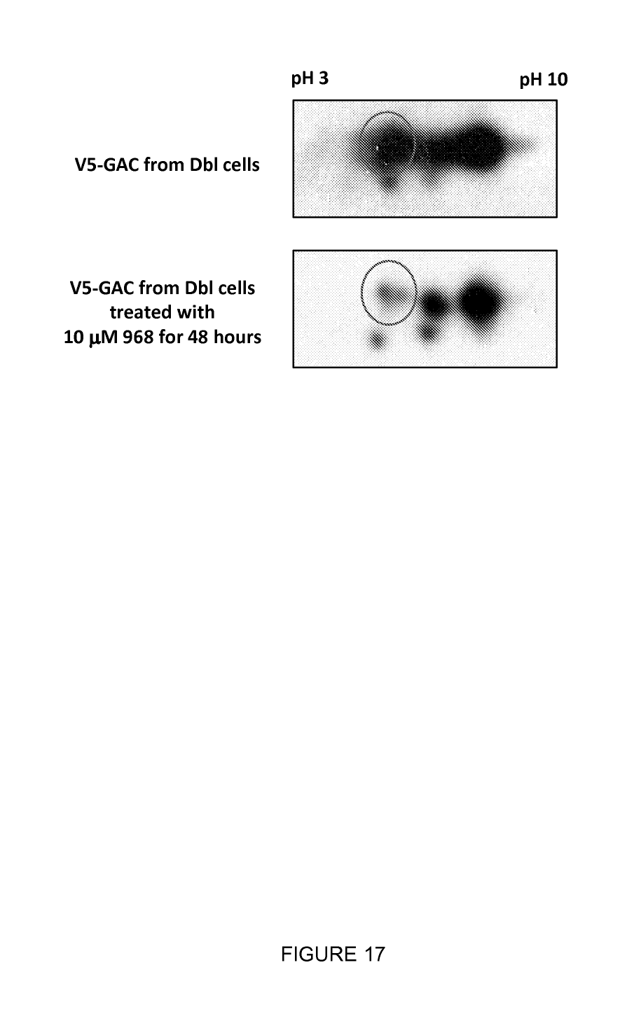

NIH 3T3 cells stably expressing the Dbl oncogene, were transiently transfected with DNA encoding a VS-tagged GAC, and then one sample was treated with 968 (10 μM) for 48 hours. The cells were then harvested and the ectopically expressed GAC was isolated by immunoprecipitation via the V5 tag. The samples were then subjected to 2-D gel analysis to separate the V5-GAC by charge and size, and the V5-tagged GAC was visualized by Western blotting using an anti-V5 antibody. The treatment of cells with 968 resulted in the significant reduction of at least one phosphorylation state of GAC. Since 968 inhibits the enzymatic activity of GAC, and the phosphorylation appears to be required for its basal enzyme activity, it appears that 968 might be functioning to inhibit glutaminase C by inhibiting the ability of at least one site on glutaminase C to become phosphorylated. See FIG. 17.

### Example 10

### Effect of Compound 968 on Oncogenic Growth

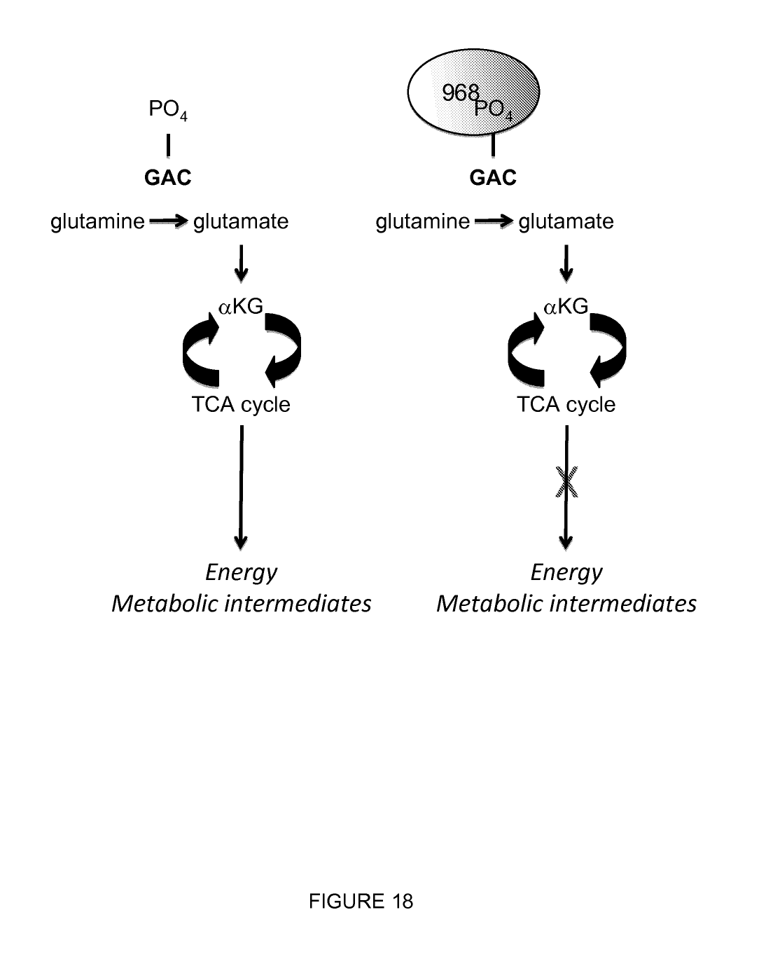

In cancer cells, GAC undergoes a phosphorylation event(s) which in not observed in nontransformed cells (left panel). This phosphorylation leads to a phosphate-independent (basal) activation of GAC, resulting in a rise in glutamate production which feeds the TCA cycle to supply the cancer cell with the energy and metabolic intermediates it needs to support tumorigenic growth. It is proposed that 968 may function by blocking a tumor-specific phosphorylation event on GAC which is necessary for its phosphate-independent activity (right panel). The inhibition of GAC reduces the influx of glutamate into the TCA cycle and, thus, effectively “starves” the tumor cell of needed energy and metabolic intermediates. See FIG. 18.

### Example 11

### Inhibition of GAC (glutaminase C) Activity by Compound 968 and BA-968

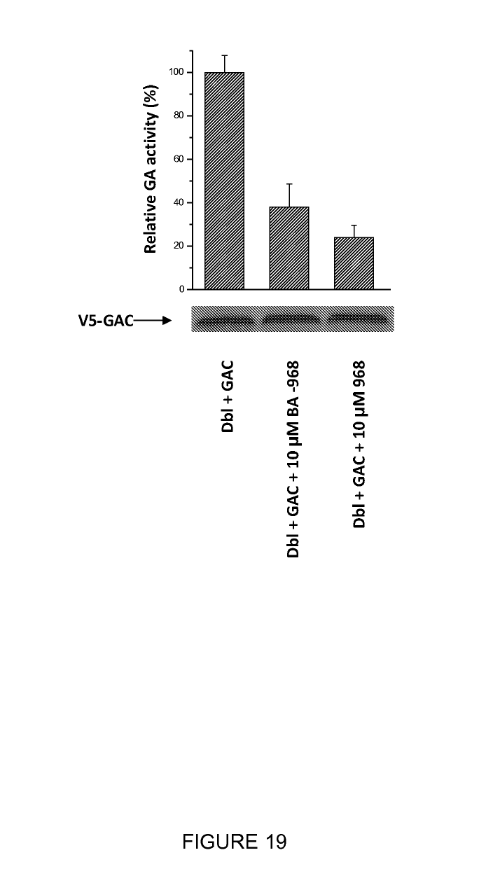

NIH 3T3 cells stably expressing oncogenic Dbl were transiently transfected with DNA encoding VS-tagged GAC, and cells were treated with either 968 or BA-968 (10 μM) as indicated for 48 hours. The cells were then harvested and the ectopically expressed GAC was isolated by immunoprecipitation via the V5 tag. See FIG. 19. The samples were then assayed for glutaminase activity in the absence of phosphate (top panel) and the relative expression levels of V5-GAC was determined by Western blotting using an anti-V5 antibody (bottom panel).

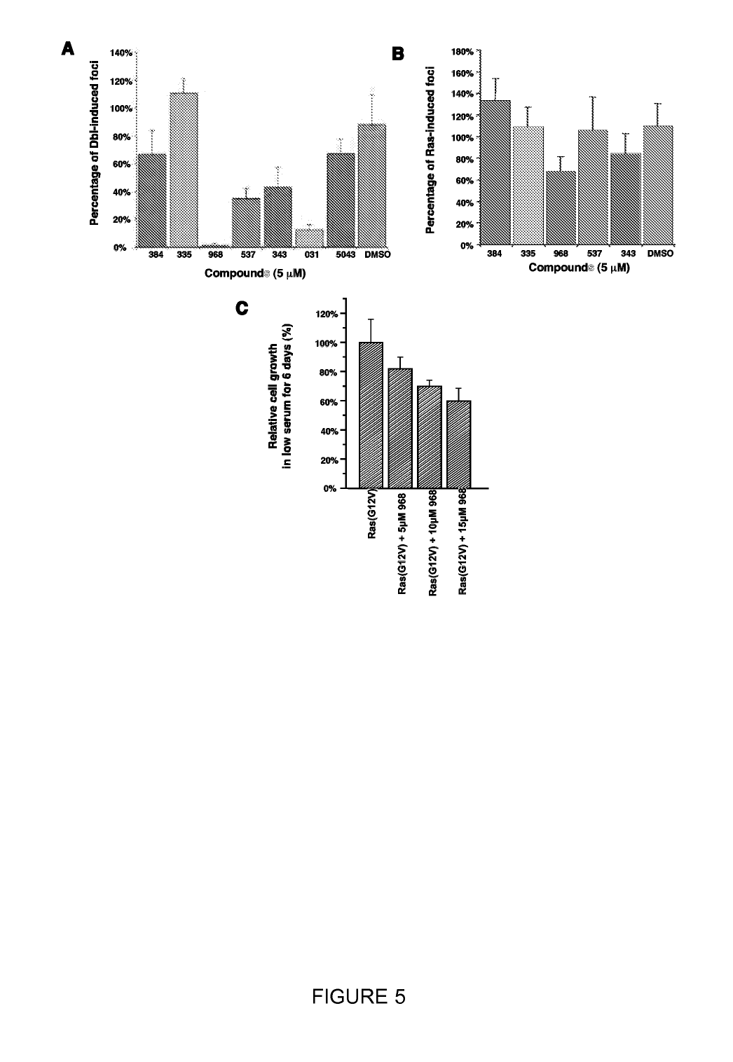

It is demonstrated here that members of the benzo[a]phenanthridinone family block the cellular transformation induced by the Rho family-GEF oncogenic Dbl (Diffuse B-cell lymphoma), as read-out in focus-forming assays or by growth in low serum (FIGS. 1A and 1B). The most effective molecule, designated 968, is active at 1-10 μM. The phenyl ring (circled in FIG. 1C) is essential for inhibitory activity, as the molecule designated BA-968 is still effective, albeit slightly less potent, in blocking Dbl-induced transformation. Compounds 335 or 384, which lack only the dimethyl amine or bromine, respectively, show little or no inhibition (FIGS. 1A and 5A). 968 is a more potent inhibitor of Dbl-induced transformation, compared to oncogenic H-Ras, when assaying focus formation in NIH 3T3 cells (FIGS. 5A and 5B), or growth in low serum (compare FIGS. 1B and 5C), indicating that the transforming activities of Rho GTPases are particularly sensitive to this small molecule. Treatment with 968 shows no significant effects on the growth or morphology of normal NIH 3T3 cells (FIGS. 1D and 1E).

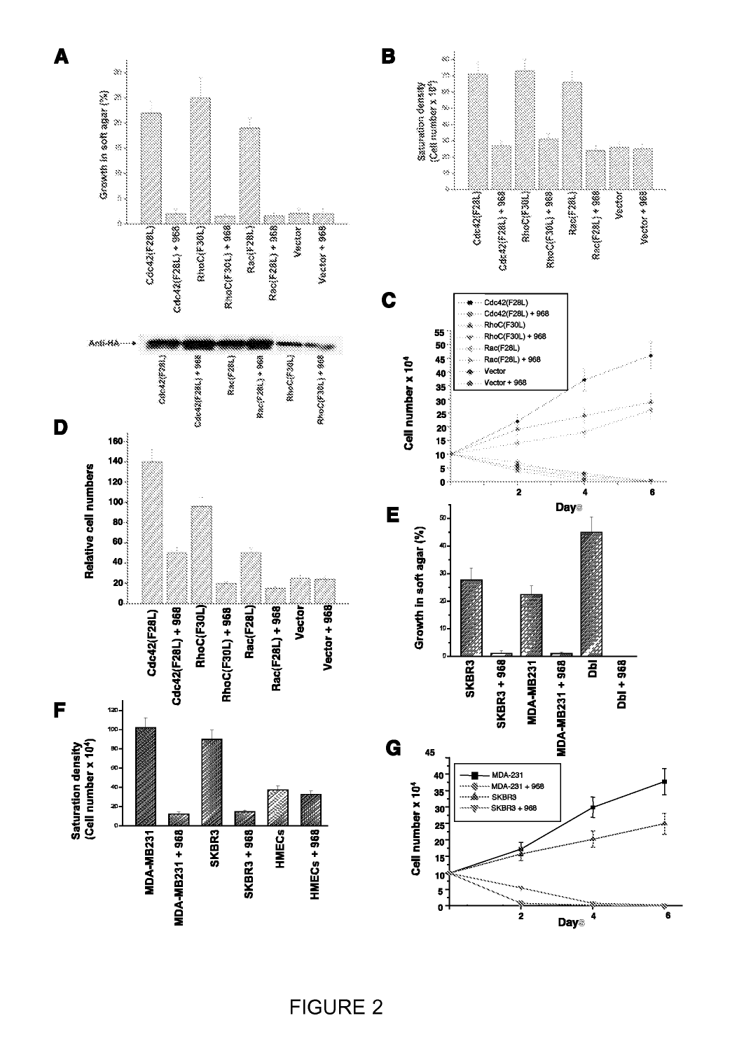

Mutated Rho GTPases that undergo constitutive GDP-GTP exchange (“fast-cyclers”) mimic many of the actions of Dbl, enabling cells to grow in low serum, form colonies in soft-agar (i.e. anchorage-independent growth), and induce tumor formation when injected into immuno-compromised mice (Lin et al., “Specific Contributions of the Small GTPases Rho, Rac and Cdc42 to Dbl Transformation,” J. Biol. Chem. 274:23633-23641 (1999), which is hereby incorporated by reference in its entirety). Cells transformed by different fast-cycling Rho GTPases were used to determine whether 968 blocked the signaling activity of a specific Rho GTPase-target of Dbl, such as RhoC. In fact, 968 inhibited the transforming activity of a number of activated Rho GTPase mutants, blocking their ability to stimulate NIH 3T3 cells to form colonies in soft-agar (FIG. 2A) and to grow to high density (FIG. 2B) or under low serum conditions (FIG. 2C), as well as inhibiting their invasive activity (FIG. 2D).

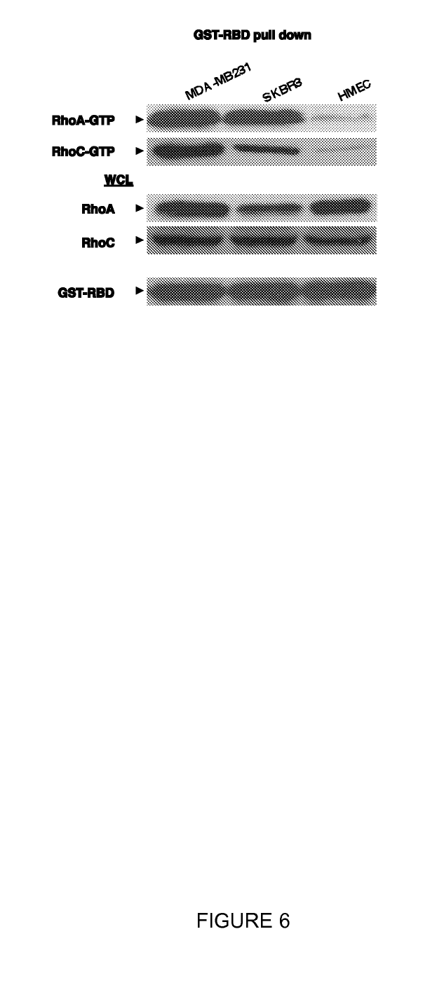

Rho GTPases have been implicated in human breast cancer (Burbelo et al., “Altered Rho GTPase Signaling Pathways in Breast Cancer Cells,” Breast Cancer Res. Treat. 84:43-48 (2004); Valastyan et al., “A Pleiotropically Acting microRNA, miR-31, Inhibits Breast Cancer Metastasis,” Cell 137:1032-1046 (2009), which are hereby incorporated by reference in their entirety). The highly invasive MDA-MB231 cells and SKBR3 cells represent two examples of breast cancer cell lines that exhibit hyper-activated RhoA and RhoC compared to normal human mammary epithelial cells (HMECs), as indicated in pull-down assays using GST fused to the Rho-binding domain of the effector protein Rhotekin (FIG. 6). Compound 968 inhibits the ability of both of these breast cancer cells to form colonies in soft agar, as effectively as it blocked Dbl-induced colony formation in NIH 3T3 cells (FIG. 2E). Similarly, 968 inhibits their growth to high density and in low serum, while having little effect on the growth of HMECs (FIGS. 2F and 2G).

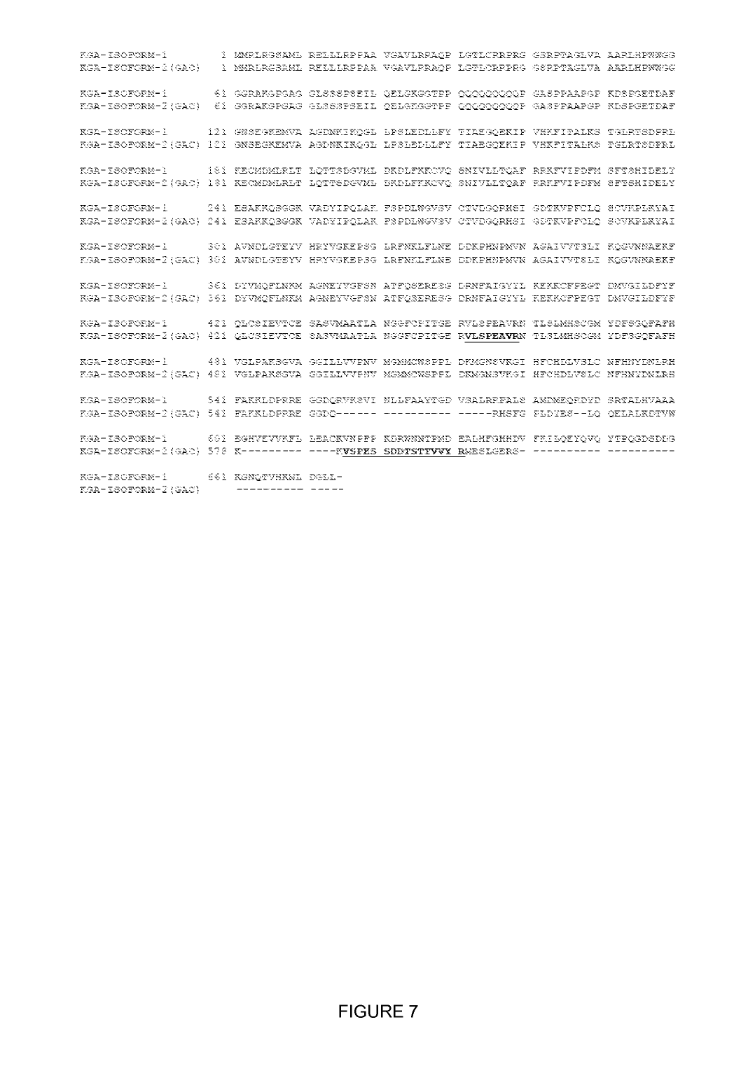

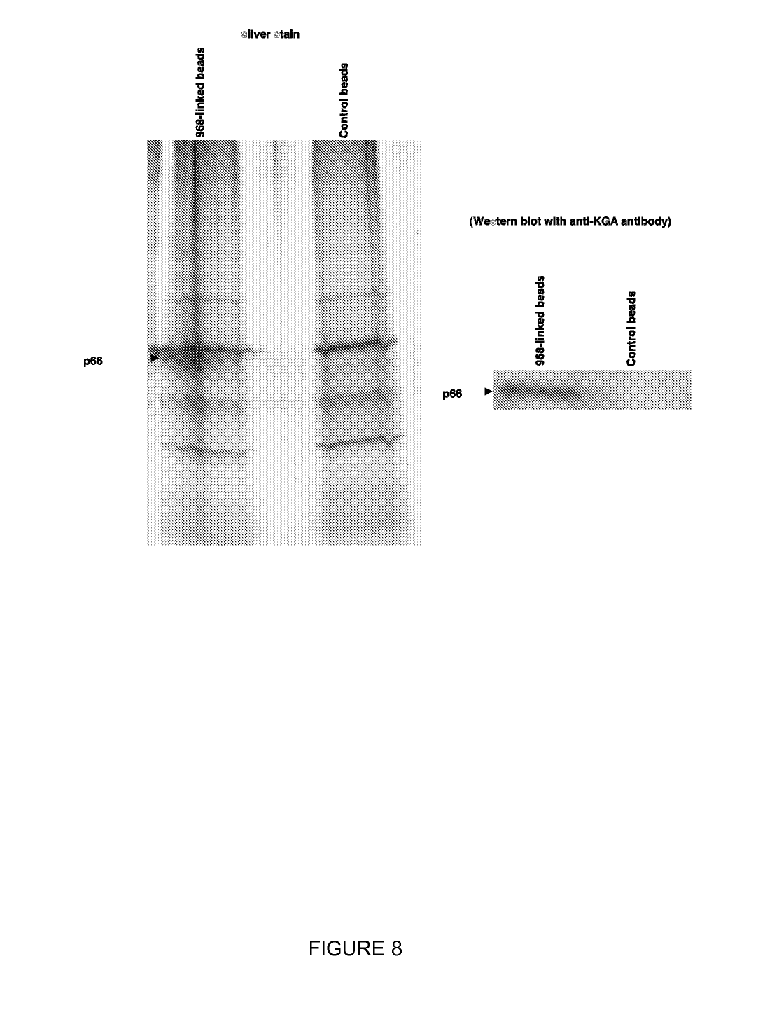

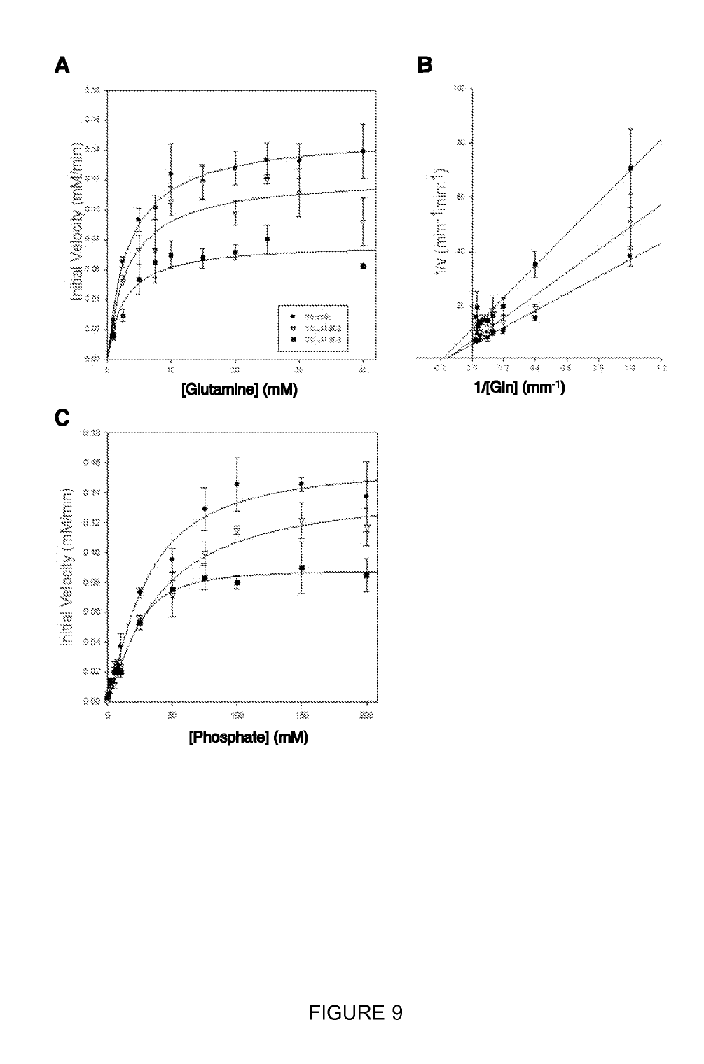

The binding target for compound 968 can be identified by using the molecule active moiety (circled in FIG. 1C) labeled with biotin in affinity precipitation experiments with streptavidin beads. This experiment leads to the detection of a silver-stained band on SDS-gels, Mᵣ ˜66 kDa, that can be isolated from Cdc42(F28L)-expressing NIH 3T3 cell lysates with the biotin-labeled 968-derivative immobilized to streptavidin beads, but not with beads alone. Microsequence analysis indicates that this 968-binding partner is the mouse isoform-2 ortholog of human glutaminase C (GAC), one of two splice variants of an enzyme found in kidney and other tissues, collectively referred to as kidney-type glutaminase (KGA), that catalyzes the hydrolysis of glutamine to glutamate and ammonium (Curthoys, N. P., “Regulation of Glutaminase Activity and Glutamine Metabolism,” Annu. Rev. Nutr. 15:133-159 (1995), which is hereby incorporated by reference in its entirety) (FIG. 7). It has been verified that the biotin-labeled active moiety of 968, when immobilized on streptavidin beads, affinity-precipitates an endogenous protein of Mᵣ ˜66 kDa that reacted with an antibody recognizing both isoforms of KGA (FIG. 8), as well as precipitated ectopically expressed VS-tagged GAC (FIG. 3A, top panel). Compound 968 inhibits the enzymatic activity of purified mouse GAC protein expressed in E. coli, whereas structurally-related compounds like 335 that are less effective at blocking Dbl-transformation (FIG. 1A), also showed little ability to inhibit enzyme activity (FIG. 3A). The inhibition by 968 is neither competitive versus substrate (glutamine) nor inorganic phosphate, an activator of the enzyme (Kenny et al., “Bacterial Expression, Purification and Characterization of Rat Kidney-Type Mitochondrial Glutaminase,” Protein Expr. Purif. 31:140-148 (2003), which is hereby incorporated by reference in its entirety), suggesting that it acts in an allosteric manner (FIGS. 9A-C).

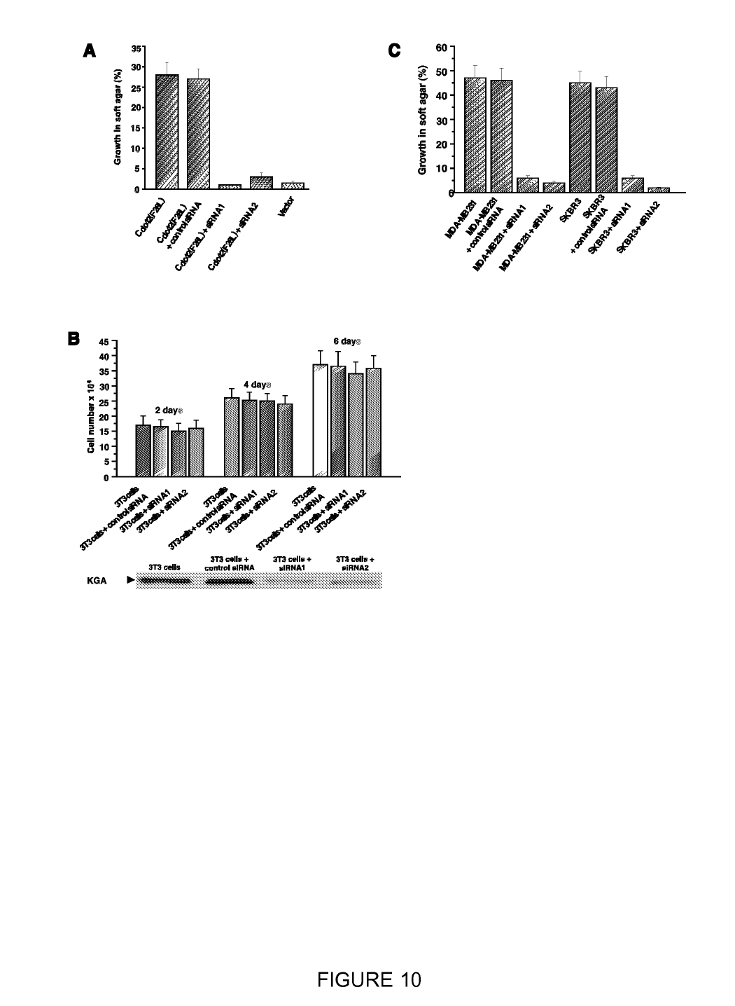

Reducing KGA expression by using siRNAs targeting both of its isoforms inhibits the ability of Cdc42(F28L) to stimulate growth in low serum (FIG. 3B) and colony formation in soft agar (FIG. 10A). Knocking-down KGA expression in control NIH 3T3 cells fails to significantly inhibit their growth in normal serum (FIG. 10B), consistent with the inability of 968 to affect their growth or overall morphology (FIGS. 1D and 1E), whereas it strongly inhibits MDA-MB231 and SKBR3 cells from growing in low serum (FIG. 3C) and in soft-agar (FIG. 10C). Because 968 blocks the growth of transformed/cancer cells by inhibiting glutaminase C activity, it should also eliminate the next step in glutamine metabolism, i.e., the generation of α-ketoglutarate from the GA-product glutamate. Moreover, this would predict that 968-inhibition can be circumvented by adding a cell-permeable analog of α-ketoglutarate to cells. Indeed, it was found to be the case in SKBR3 cells when assaying growth in low serum (FIG. 3D), as well as in Dbl-transformed cells when assaying focus-formation (FIG. 3E).

Dbl-transformed fibroblasts exhibit much higher basal GA activity (i.e. assayed in the absence of inorganic phosphate) than non-transformed NIH 3T3 cells (FIG. 3F). Cdc42(F28L)-expressing cells show basal levels of GA activity that are lower than those for Dbl-transformed cells, but still higher than non-transformed cells. The GA activity in control NIH 3T3 cells is strongly stimulated by phosphate (−6-fold), such that it approaches the maximum phosphate-stimulated activity obtained in transformed cells (FIG. 11A). Treatment of transformed cells with 968 inhibits their GA activity, with the basal activity being more sensitive than the phosphate-stimulated activity (see FIGS. 3F and 11A).

Both MDA-MB231 and SKBR3 cells show significantly higher basal GA activity, compared to normal HMECs, that is sensitive to 968 (FIG. 3G, top panel; the bottom panel shows that equivalent amounts of mitochondrial protein were assayed, by using the mitochondrial marker VDAC/Porin (Shimizu et al., “Bc1-2 Family Proteins Regulate the Release of Apoptogenic Cytochrome c by the Mitochondrial Channel VDAC,” Nature 399:483-487 (1999), which is hereby incorporated by reference in its entirety)). Inorganic phosphate strongly stimulates the GA activity in HMECs (˜5-fold), and although it is still lower than the maximum activity measured in MDA-MB231 cells, it is similar to the phosphate-stimulated activity assayed in SKBR3 cells (FIG. 11B). Knock-downs of RhoA and RhoC in SKBR3 cells markedly reduce their basal GA activity, without significantly affecting the direct stimulation of the enzyme by phosphate, indicating that the basal enzyme activity in these breast cancer cells is Rho GTPase-dependent (FIG. 11C).

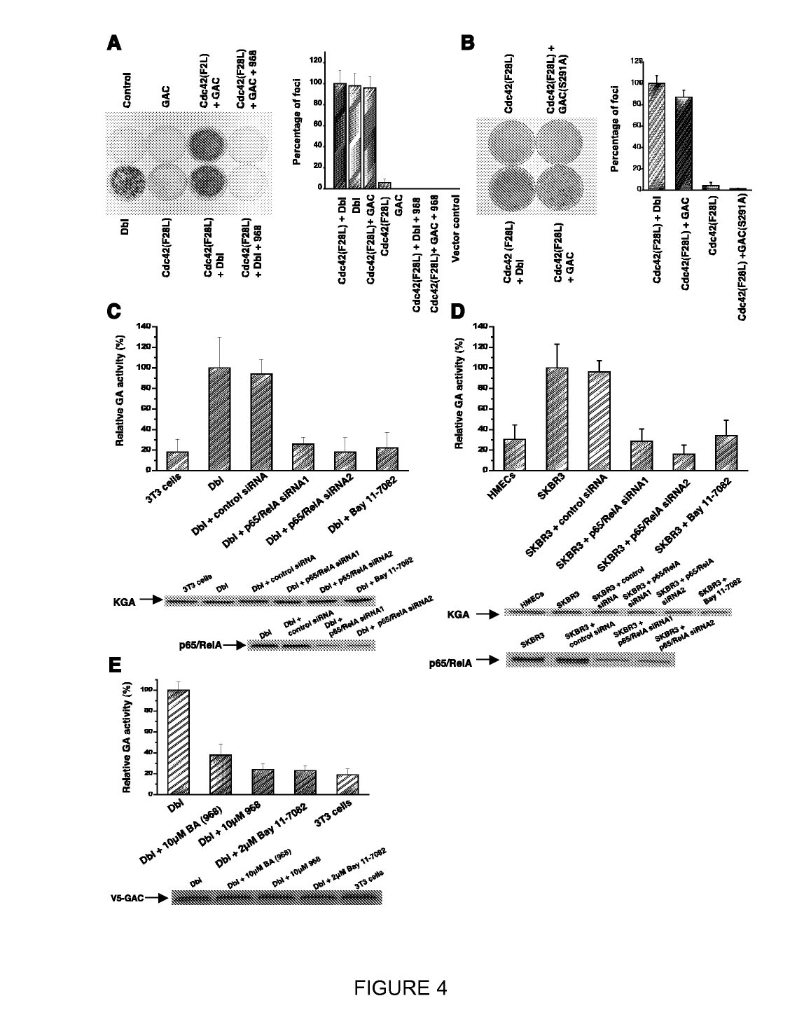

The expression of GAC is shown to be significantly increased in B-lymphoma and prostate cancer cells and to be necessary for their proliferation and survival (Gao et al., “c-Myc Suppression of miR-23a/b Enhances Mitochondrial Glutaminase Expression and Glutamine Metabolism,” Nature 458:762-765 (2009), which is hereby incorporated by reference in its entirety). The ectopic expression of GAC alone is insufficient to transform cells (FIG. 4A). However, the transient expression of GAC in cells stably expressing Cdc42(F28L) causes a dramatic increase in focus-forming activity, that matches Dbl-transformed cells which typically exhibit large numbers of foci and high basal levels of GA activity, and can be blocked by treatment with 968. When the catalytically dead GAC(S291A) mutant is co-expressed with Cdc42(F28L), there is no detectable increase in transforming activity compared to that for Cdc42(F28L) alone (FIG. 4B). Collectively, these findings demonstrate the need to reach a threshold level of GA activity to achieve the maximum transforming signal and that increased GAC expression alone is not sufficient for increased basal activity.

MDA-MB231 breast cancer cells show higher KGA expression compared to SKBR3 cells or normal HMECs when using an antibody which recognizes both enzyme isoforms (FIG. 3C, bottom panel; FIG. 3G, bottom panels), which likely accounts for their increased levels of basal (FIG. 3G, top panel) and phosphate-stimulated GA activity (FIG. 11B). However, significant differences in KGA expression in Dbl- or Cdc42(F28L)-transformed cells compared to control cells (FIG. 3F, bottom panel) have not been detected, consistent with their showing similar levels of phosphate-stimulated GA activity (FIG. 11A). Likewise, KGA expression in SKBR3 cells is not significantly different from normal HMECs (FIG. 3G, bottom panels), as born out by their similar levels of phosphate-stimulated GA activity (FIG. 11B). Therefore, the increase in basal GA activity in SKBR3 cells, as well as in Dbl- and Rho GTPase-transformed fibroblasts, cannot be simply attributed to an up-regulation of enzyme expression.

A clue regarding how GA is activated in these transformed/cancer cells came from the finding that the treatment of Dbl-transformed cells and SKBR3 breast cancer cells with BAY 11-7082, which blocks NF-κB activation by inhibiting the upstream kinase IKKB (Pickering et al., “Pharmacological Inhibitors of NF-κB Accelerate Apoptosis in Chronic Lymphocytic Leukemia Cells,” Oncogene 26:1166-1177 (2007), which is hereby incorporated by reference in its entirety), significantly reduces their basal GA activity (FIGS. 4C and 4D, respectively). NF-κB is activated by Dbl and various Rho GTPases (Perona et al., “Activation of the Nuclear Factor-κB by Rho, CDC42, and Rac-1 Proteins,” Genes Dev. 11:463-475 (1997); Joyce et al., “Integration of Rac-Dependent Regulation of cyclin D1 Transcription Through a Nuclear Factor-κB-Dependent Pathway,” J. Biol. Chem. 274:25245-25249 (1999); Cammarano et al., “Dbl and the Rho GTPases Activate NFκB by IκB kinase (IKK)-Dependent and IKK-Independent Pathways,” J. Biol. Chem. 276:25876-25882 (2001), which are hereby incorporated by reference in their entirety), and is essential for Dbl-transformation (Whitehead et al., “Dependence of Dbl and Dbs Transformation on MEK and NF-kappaB Activation,” Mol. Cell. Biol. 19:7759-7770 (1999), which is hereby incorporated by reference in its entirety) and for the transformed phenotypes of human breast cancer cells (Sovak et al., “Aberrant Nuclear Factor-κB/Rel Expression and the Pathogenesis of Breast Cancer,” J. Clin. Invest. 100:2952-2960 (1997), which is hereby incorporated by reference in its entirety). Knocking-down the p65/RelA subunit of NF-κB in Dbl-transformed cells and SKBR3 cells also markedly reduces their basal GA activity (FIGS. 4C and 4D), whereas treatment with BAY11-7082 or knock-downs of p65/RelA has little or no effect on the direct stimulation of the enzyme by phosphate (Figures S7D and 11E).

NF-κB might regulate GA by inducing the expression of a protein that stimulates its activity through a direct interaction or via a post-translational modification. The latter would be analogous to how the tyrosine phosphorylation of the M2 isoform of pyruvate kinase has been suggested to influence glycolysis in cancer cells (Christofk et al., “Pyruvate Kinase M2 is a Phosphotyrosine-Binding Protein,” Nature 452:181-186 (2008), which is hereby incorporated by reference in its entirety). Indeed, it has been found that VS-tagged GAC, when ectopically expressed in Dbl-transformed cells followed by its immunoprecipitation (IP), exhibits significantly higher activity compared to V5-GAC IPed from non-transformed NIH 3T3 cells (FIG. 4E). The GA activity IPed from Dbl-transformed cells is inhibited by both 968 and BA-968, and is markedly reduced when NF-κB activation is blocked prior to IP, thus consistent with the suggestion that GAC is modified in transformed cells in an NF-κB-dependent manner.

The importance of cellular metabolism in the development of cancer, and in particular, the early observations that tumor cells exhibit enhanced glycolytic activity (i.e. the “Warburg effect”), are receiving renewed attention (DeBerardinis et al., “Beyond Aerobic Glycolysis Transformed Cells Can Engage in Glutamine Metabolism that Exceeds the Requirement for Protein and Nucleotide Synthesis,” Proc. Natl. Acad. Sci. USA 104:19345-19350 (2007); Christofk et al., “Pyruvate Kinase M2 is a Phosphotyrosine-Binding Protein,” Nature 452:181-186 (2008), which are hereby incorporated by reference in their entirety). ¹³C-NMR metabolic flux experiments have demonstrated that while proliferating cancer cells exhibit a pronounced Warburg effect, their TCA cycle remains intact and is driven by glutamine metabolism (DeBerardinis et al., “Beyond Aerobic Glycolysis: Transformed Cells Can Engage in Glutamine Metabolism that Exceeds the Requirement for Protein and Nucleotide Synthesis,” Proc. Natl. Acad. Sci. USA 104:19345-19350 (2007), which is hereby incorporated by reference in its entirety). This enables cancer cells to supply a significant fraction of TCA cycle intermediates as precursors for biosynthetic pathways (DeBerardinis et al., “The Biology of Cancer: Metabolic Reprogramming Fuels Cell Growth and Proliferation,” Cell Metab. 7:11-19 (2008), which is hereby incorporated by reference in its entirety), and is consistent with the observations that tumor cells exhibit increased rates of glutamine metabolism and consume greater amounts of glutamine compared to normal cells (Medina et al., “Relevance of Glutamine Metabolism to Tumor Cell Growth,” Mol. Cell. Biochem. 113:1-15 (1992), which is hereby incorporated by reference in its entirety). The observation that different transformed cell lines and cancer cells show elevated GA activity in their mitochondria that is dependent on Rho GTPase/NF-κB-signaling provides a mechanism for how these demands for elevated glutamine metabolism are met. Moreover, the ability of the small molecule 968 to inhibit GA activity and influence the aberrant growth properties of transformed/cancer cells raise intriguing possibilities for new strategies of therapeutic intervention against cancer.

### Example 12

### Identification of the Activating Phosphorylation Site of Glutaminase C (GAC)

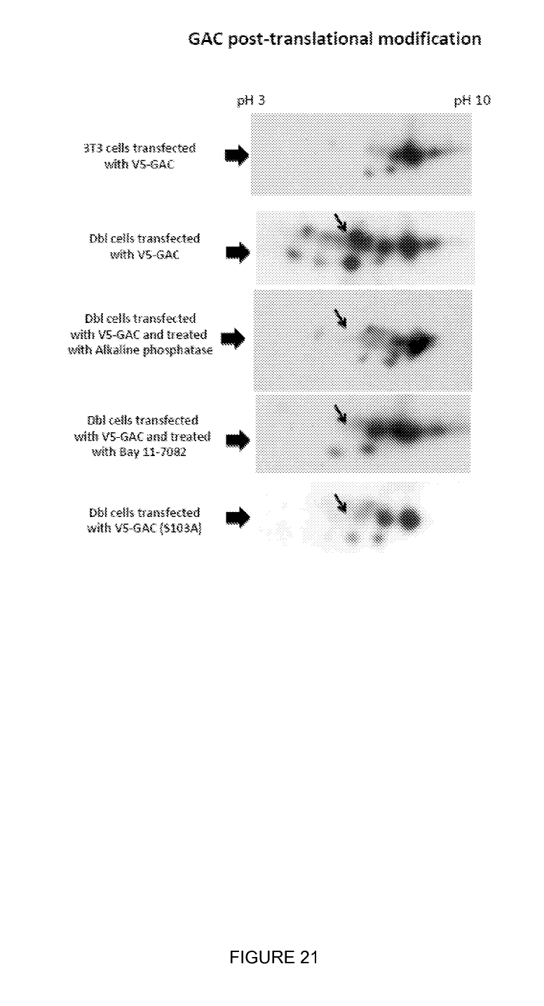

GAC runs distinctly on a 2D gel depending whether it is expressed in normal (untransformed) NIH 3T3 cells or in NIH 3T3 cells which are transformed by the Dbl oncogene. Specifically, there is one major species of GAC present in NIH 3T3 cells whereas in Dbl cells, two additional species of GAC appear which migrate with a more negative charge but at the same molecular mass as the original species (FIG. 21, first and second panels). Treatment of GAC isolated from Dbl cells with alkaline phosphatase prior to 2D gel analysis results in the appearance of only a single GAC species, indicating that the variant species of GAC are the result of a phosphorylation(s) (FIG. 21, third panel). Additionally, treatment of Dbl cells with an NF-kB inhibitor, Bay 11-7082 (FIG. 20), for 48 hours results in loss of the most negative migrating major species of GAC (FIG. 21, fourth panel—note, the most negative major species of GAC is indicated with an arrow). Glutaminase activity assays on GAC isolated under identical conditions as to those of the 2D gel analysis shown in FIG. 21, top four panels, show that GAC is only active under conditions that allow for the formation of the most negative major species of GAC, thus leading to the idea that a phosphorylation event on GAC is necessary to create an active enzyme in vivo. Additionally, it has been shown that small molecules of the 968 family which are able to inhibit GAC in vivo do so by blocking the ability of GAC to become phosphorylated. Thus, it was of great interest to identify the amino acid(s) on GAC whose phosphorylation is causal in the activation of this enzyme.

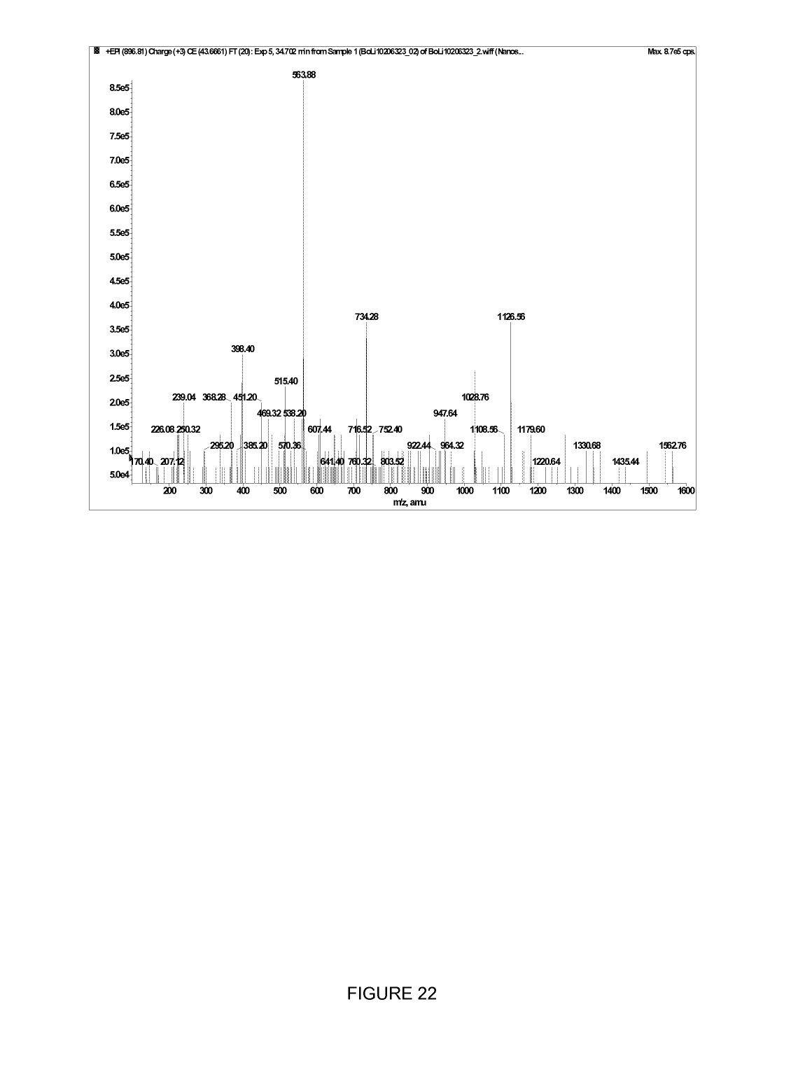

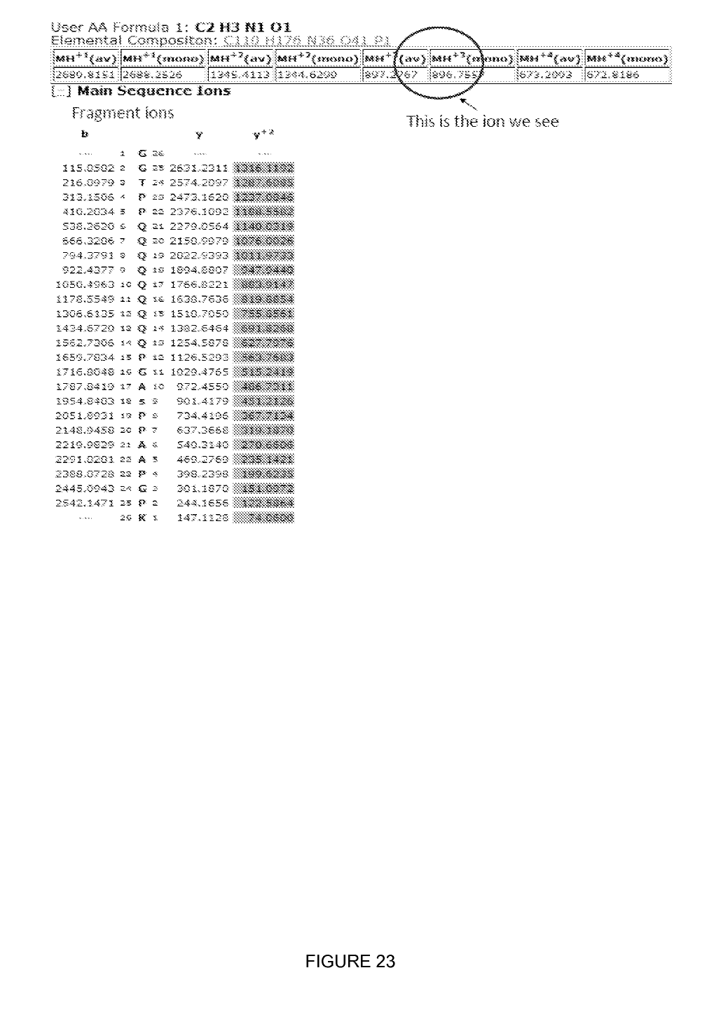

To this end, a VS-tagged form of GAC was expressed in Dbl cells, isolated by immunoprecipitation and then was subjected to 2D gel analysis. After 2D gel analysis, the GAC migrating as the most negative species was isolated, digested, and analyzed by mass spectrometry at Cornell University's Proteomics and Mass Spectrometry facility. A single phosphorylated amino acid was detected (FIG. 22) corresponding to Serine 103 on mouse GAC (which is equivalent to Serine 95 on human GAC) within the isolated peptide: GGTPPQQQQQQQQQPGAS*PPAAPGPK (SEQ ID NO: 7, FIG. 23).

To verify that Serine 103 was indeed phosphorylated on GAC expressed in Dbl cells, a phospho-defective GAC mutant (GAC S103A) was generated. When GAC (S103A) was isolated from Dbl cells and subjected to 2D gel analysis, the most negatively migrating species of GAC was not observed (FIG. 21, bottom panel). These data suggest that Serine 103 on mouse GAC is a physiologically relevant phosphorylation site.

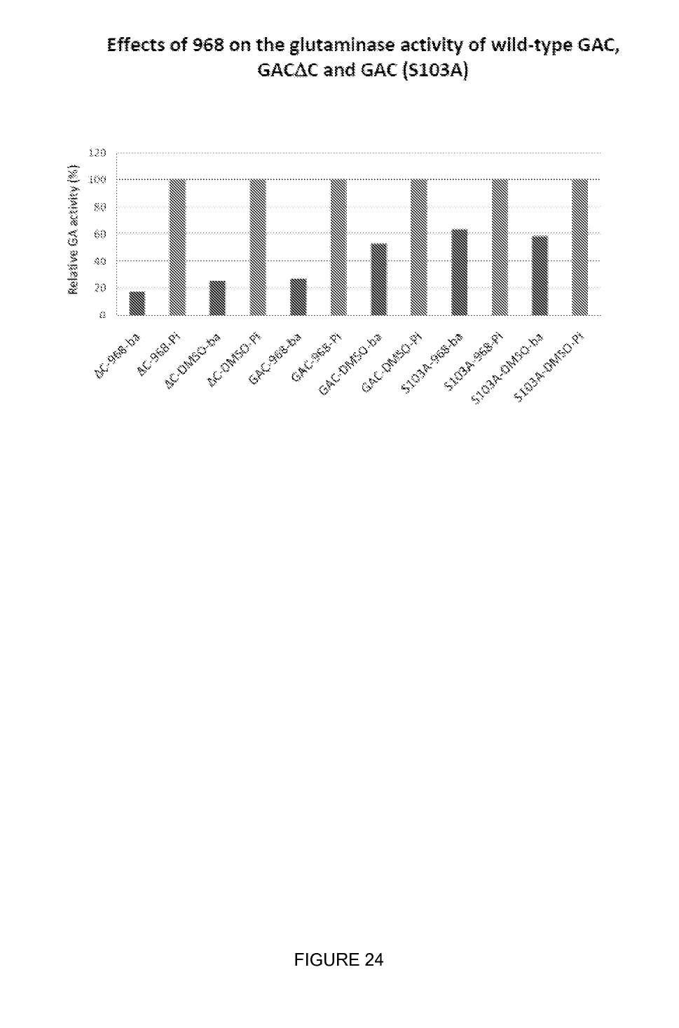

Next the effect of the substitution of an alanine for a serine at amino acid 103 on the enzymatic activity of GAC was examined, as well as its sensitivity to the small molecule, 968. Dbl-transformed cells were transfected with cDNA coding for VS-tagged forms of wild-type GAC, GAC (S103A), and a C-terminal truncation mutant of GAC that has been found to have impaired glutaminase activity (GACΔC). Following transfection, the cells were treated for 48 hours with 968 (10 μM) and then harvested. The different GAC proteins were then isolated by immunoprecipitation and assayed for their glutaminase activity in the absence (i.e., basal activity) and presence of phosphate (FIG. 24). As expected, wild-type GAC showed significant basal glutaminase activity that was susceptible to treatment with 968. The basal activity of GACΔC was approximately one third of wild type GAC and this activity was not further reduced by the treatment of cells with 968. Interestingly, the glutaminase activity of GAC (S103A) was slightly elevated when compared to wild type GAC, and was not affected by 968 treatment. Initially, the high glutaminase activity of the GAC (S103A) mutant was surprising as it had been predicted that this phospho-defective mutant should have a reduced enzymatic activity. It appears that Serine 103 must be involved in making an important hydrogen bond through its hydroxyl moiety which plays a role in keeping the enzyme in the inactive state. When this hydrogen bond is broken, either by mutating the serine to an alanine or upon the phosphorylation of this serine residue, GAC transitions to an active conformation. The observation that 968 does not effect the glutaminase activity of GAC (S103A) suggests that the mutant is constitutively active since other studies have indicated that 968 works by maintaining the inactive state of the enzyme rather than causing a direct inhibition of the active species. Thus, Serine 103 plays a critical role in the regulation of the glutaminase activity of GAC.

### Example 13

### mTOR Phosphorylates Glutaminase C (GAC)

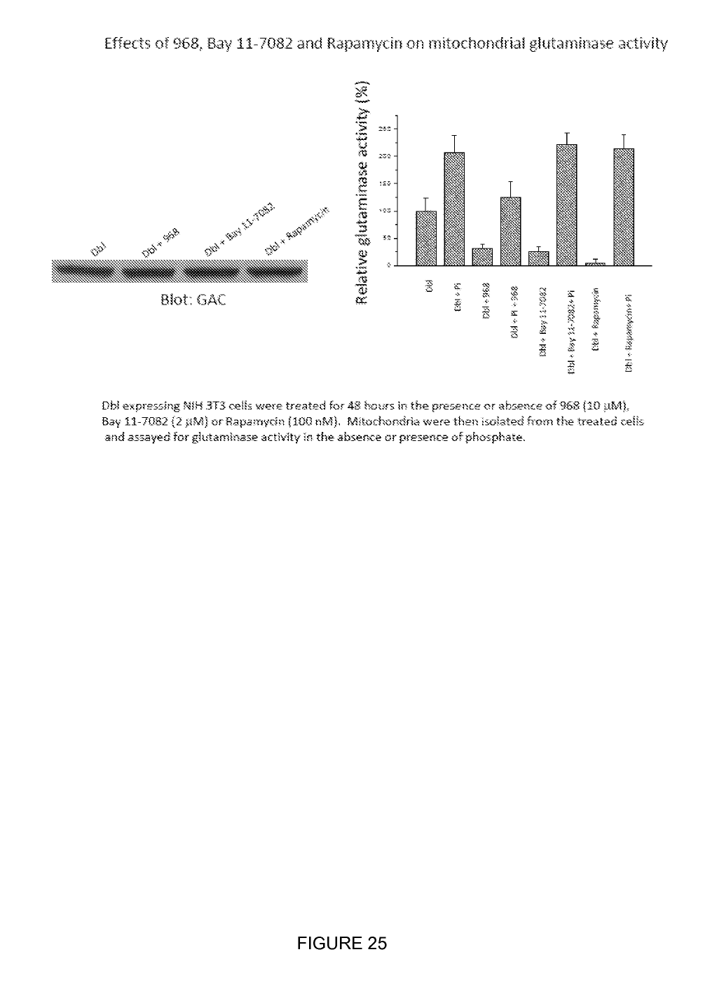

Having identified a critical phosphorylation site on GAC, the next goal was to identify the kinase which is responsible for this phosphorylation. In addition to treating cells with the known GAC inhibitor, 968, cells were also treated with an inhibitor of NF-κB (Bay 11-7082, FIG. 20) and an inhibitor of mTOR (rapamycin) for 48 hours, and then the glutaminase activity of GAC was examined. As can be seen in FIG. 25, the addition of both the NF-κB inhibitor and the mTOR inhibitor caused a significant loss of glutaminase activity suggesting that these proteins play a role in the regulation of GAC.

mTOR is a protein kinase which plays an important role in the cell as a master signal integrator. As part of a larger complex, mTORC1, mTOR is able to sense the status of growth factors, nutrients and energy in the cell, and, if conditions are sufficient, it signals for cell growth. The most appreciated way this is achieved is through the regulation of translational events by mTORC1. However, other roles for mTOR have been surfacing, including a role for mTOR in the mitochondria. When the localization of mTOR in Dbl-transformed cells transfected with wild-type GAC was examined, some amount of mTOR co-localizing in the mitochondria with GAC was found. However, when Dbl-transformed cells expressed the C-terminal truncated form of GAC, GACΔC, the mitochondrial localization of mTOR was lost. GACΔC, itself does not localize properly to the mitochondria. Thus there appears to be a correlation between the cellular distribution of GAC and mTOR localization, which in turn suggests a physical interaction between these two proteins.

To test this, Dbl-transformed cells were transiently transfected with wild-type GAC, GACΔC and GAC (S103A), and then these proteins were isolated from cell lysates by immunoprecipitation. Using an mTOR antibody, we then probed Western blots of these immunoprecipitations to determine if mTOR was able to associate with these GAC proteins. mTOR can indeed be found in a complex with wild-type GAC and GACΔC. However, when Serine 103 of GAC is mutated to alanine, the interaction with mTOR is lost. Additionally, when radio-labeled ATP is added to the immunoprecipitated proteins under conditions favorable for the activation of the mTOR kinase, the phosphorylation of proteins immunoprecipitated with wild-type GAC and GACΔC is observed, but not for proteins immunoprecipitated with GAC (S103A). Together, these data demonstrate that mTOR associates with GAC through Serine 103. Because mTOR can associate with GAC, and mTOR kinase activity is necessary for the activation of GAC, it is possible that mTOR is functioning as a GAC kinase, phosphorylating GAC at Serine 103 to activate the enzyme.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of reducing production of glutamate from glutamine in a cell or a tissue, said method comprising:
inhibiting activating phosphorylation within the amino acid sequence of SEQ ID NO: 1 of glutaminase C in the cell or tissue under conditions effective to reduce production of glutamate from glutamine.

2. The method according to claim 1, wherein the amino acid residue X of SEQ ID NO: 1 is valine or alanine.

3. The method according to claim 1, wherein the activating phosphorylation event occurs at serine 95 of the amino acid sequence of SEQ ID NO: 2 or at serine 103 of the amino acid sequence of SEQ ID NO: 3.

4. The method according to claim 1, wherein said inhibiting the activating phosphorylation within the amino acid sequence of SEQ ID NO: 1 of glutaminase C is carried out by inhibiting a transcription factor which regulates phosphorylation of glutaminase C or a kinase which phosphorylates glutaminase C.

5. The method according to claim 4, wherein the kinase is mTOR.

6. The method according to claim 5, wherein mTOR is inhibited by rapamycin or its homologs.

7. The method according to claim 4, wherein the transcription factor is NF-κB.

8. The method according to claim 7, wherein NF-κB is inhibited by BAY 11-7082.

9. A method of treating or preventing a condition mediated by activating phosphorylation of glutaminase C in a subject, said method comprising:
selecting a subject having or being susceptible to a condition mediated by the activating phosphorylation within the amino acid sequence of SEQ ID NO: 1 of glutaminase C and
administering to said selected subject an inhibitor of the activating phosphorylation within the amino acid sequence of SEQ ID NO: 1 of glutaminase C activity under conditions effective to treat or prevent the condition mediated by the activating phosphorylation of glutaminase C.

10. The method according to claim 9, wherein the amino acid residue X of SEQ ID NO: 1 is valine or alanine.

11. The method according to claim 9, wherein the method is for treating a condition mediated by the activating phosphorylation of glutaminase C.

12. The method according to claim 9, wherein the method is for preventing a condition mediated by the activating phosphorylation of glutaminase C.

13. The method according to claim 9, wherein the inhibitor inhibits phosphorylation of serine 95 of the amino acid sequence of SEQ ID NO: 2 or serine 103 of the amino acid sequence of SEQ ID NO: 3.

14. The method according to claim 9, wherein said inhibitor of the activating phosphorylation within the amino acid sequence of SEQ ID NO: 1 of glutaminase C inhibits a transcription factor which regulates phosphorylation of glutaminase C or a kinase which phosphorylates glutaminase C.

15. The method according to claim 14, wherein the kinase is mTOR.

16. The method according to claim 15, wherein mTOR is inhibited by rapamycin or its homologs.

17. The method according to claim 14, wherein the transcription factor is NF-κB

18. The method according to claim 17, wherein NF-κB is inhibited by BAY 11-7082.

19. The method of claim 9, wherein the condition is cancer.

20. The method according to claim 19, wherein the cancer is breast cancer, lung cancer, brain cancer, pancreatic cancer, or colon cancer.

21. The method of claim 9, wherein said administering is carried out parenterally, orally, subcutaneously, intravenously, intramuscularly, extraperitoneally, by intranasal instillation, or by application to mucous membranes.

22. A method of detecting a condition mediated by activating phosphorylation within the amino acid sequence of SEQ ID NO: 1 of glutaminase C, said method comprising:
providing a cell or tissue;
providing a reagent that specifically recognizes the activating phosphorylation within the amino acid sequence of SEQ ID NO: 1 of glutaminase C;
contacting the cell or tissue with the reagent under conditions effective for the reagent to bind to phosphorylated glutaminase C and form a phosphorylated glutaminase C-reagent conjugate; and
identifying presence of the phosphorylated glutaminase C-reagent conjugate wherein the formation of the phosphorylated glutaminase C-reagent conjugate indicates the existence of a condition mediated by the activating phosphorylation of glutaminase C.

23. The method according to claim 22, wherein the amino acid residue X of SEQ ID NO: 1 is valine or alanine.

24. The method according to claim 22, wherein the reagent is an antibody.

25. The method according to claim 22, wherein the activating phosphorylation event occurs at serine 95 of the amino acid sequence of SEQ ID NO: 2 or at serine 103 of the amino acid sequence of SEQ ID NO: 3.

26. The method of claim 22, wherein the condition is cancer.

27. The method according to claim 26, wherein the cancer is breast cancer, lung cancer, brain cancer, pancreatic cancer, or colon cancer.

28. A method of screening for compounds capable of treating or preventing cancer, said method comprising:
providing a cancer cell or cancer tissue under conditions effective to produce glutamate from glutamine as a result of glutaminase C activity;
providing a plurality of candidate compounds;
contacting the cancer cell or cancer tissue with the candidate compounds under conditions effective for activating phosphorylation; and
identifying the candidate compounds which inhibit the activating phosphorylation of glutaminase C within the amino acid sequence of SEQ ID NO: 1 as a result of said contacting as having potential capability of treating or preventing cancer.

29. The method according to claim 28, wherein the amino acid residue X of SEQ ID NO: 1 is valine or alanine.

30. The method according to claim 28, wherein the activating phosphorylation occurs at serine 95 of the amino acid sequence of SEQ ID NO: 2 or at serine 103 of the amino acid sequence of SEQ ID NO: 3.

31. The method according to claim 28, further comprising:
lysing the cancer cell or cancer tissue after said contacting and before said identifying, wherein said identifying involves using an antibody that binds to phosphorylated glutaminase C.

32. The method according to claim 28, wherein said cancer cell or cancer tissue is cultured in the presence of radiolabelled phosphate and the activating phosphorylation of glutaminase C is determined by detecting radiolabelled glutaminase C.

33. The method according to claim 28, wherein the cancer is breast cancer, lung cancer, brain cancer, pancreatic cancer, or colon cancer.


SEQUENCE LISTING (7 sequences)

SEQ ID NO: 1 - protein, 10 aa, Artificial
  Consensus
QPGXSPPAAP

SEQ ID NO: 2 - protein, 598 aa, Homo sapiens
MMRLRGSGMLRDLLLRSPAGVSATLRRAQPLVTLCRRPRGGGRPAAGPAAAARLHPWWGGGGWPAEPLARGLSSSPSEILQELGKGSTHPQPGVSPPAAPAAPGPKDGPGETDAFGNSEGKELVASGENKIKQGLLPSLEDLLFYTIAEGQEKIPVHKFITALKSTGLRTSDPRLKECMDMLRLTLQTTSDGVMLDKDLFKKCVQSNIVLLTQAFRRKFVIPDFMSFTSHIDELYESAKKQSGGKVADYIPQLAKFSPDLWGVSVCTADGQRHSTGDTKVPFCLQSCVKPLKYAIAVNDLGTEYVHRYVGKEPSGLRFNKLFLNEDDKPHNPMVNAGAIVVTSLIKQGVNNAEKFDYVMQFLNKMAGNEYVGFSNATFQSERESGDRNFAIGYYLKEKKCFPEGTDMVGILDFYFQLCSIEVTCESASVMAATLANGGFCPITGERVLSPEAVRNTLSLMHSCGMYDFSGQFAFHVGLPAKSGVAGGILLVVPNVMGMMCWSPPLDKMGNSVKGIHFCHDLVSLCNFHNYDNLRHFAKKLDPRREGGDQRHSFGPLDYESLQQELALKETVWKKVSPESNEDISTTVVYRMESLGEKS

SEQ ID NO: 3 - protein, 603 aa, Mus musculus
MMRLRGSAMLRELLLRPPAAVGAVLRRAQPLGTLCRRPRGGSRPTAGLVAAARLHPWWGGGGRAKGPGAGGLSSSPSEILQELGKGGTPPQQQQQQQQQPGASPPAAPGPKDSPGETDAFGNSEGKEMVAAGDNKIKQGLLPSLEDLLFYTIAEGQEKIPVHKFITALKSTGLRTSDPRLKECMDMLRLTLQTTSDGVMLDKDLFKKCVQSNIVLLTQAFRRKFVIPDFMSFTSHIDELYESAKKQSGGKVADYIPQLAKFSPDLWGVSVCTVDGQRHSIGDTKVPFCLQSCVKPLKYAIAVNDLGTEYVHRYVGKEPSGLRFNKLFLNEDDKPHNPMVNAGAIVVTSLIKQGVNNAEKFDYVMQFLNKMAGNEYVGFSNATFQSERESGDRNFAIGYYLKEKKCFPEGTDMVGILDFYFQLCSIEVTCESASVMAATLANGGFCPITGERVLSPEAVRNTLSLMHSCGMYDFSGQFAFHVGLPAKSGVAGGILLVVPNVMGMMCWSPPLDKMGNSVKGIHFCHDLVSLCNFHNYDNLRHFAKKLDPRREGGDQRHSFGPLDYESLQQELALKDTVWKKVSPESSDDTSTTVVYRMESLGERS

SEQ ID NO: 4 - protein, 674 aa, Mus musculus
MMRLRGSAMLRELLLRPPAAVGAVLRRAQPLGTLCRRPRGGSRPTAGLVAAARLHPWWGGGGRAKGPGAGGLSSSPSEILQELGKGGTPPQQQQQQQQQPGASPPAAPGPKDSPGETDAFGNSEGKEMVAAGDNKIKQGLLPSLEDLLFYTIAEGQEKIPVHKFITALKSTGLRTSDPRLKECMDMLRLTLQTTSDGVMLDKDLFKKCVQSNIVLLTQAFRRKFVIPDFMSFTSHIDELYESAKKQSGGKVADYIPQLAKFSPDLWGVSVCTVDGQRHSIGDTKVPFCLQSCVKPLKYAIAVNDLGTEYVHRYVGKEPSGLRFNKLFLNEDDKPHNPMVNAGAIVVTSLIKQGVNNAEKFDYVMQFLNKMAGNEYVGFSNATFQSERESGDRNFAIGYYLKEKKCFPEGTDMVGILDFYFQLCSIEVTCESASVMAATLANGGFCPITGERVLSPEAVRNTLSLMHSCGMYDFSGQFAFHVGLPAKSGVAGGILLVVPNVMGMMCWSPPLDKMGNSVKGIHFCHDLVSLCNFHNYDNLRHFAKKLDPRREGGDQRVKSVINLLFAAYTGDVSALRRFALSAMDMEQRDYDSRTALHVAAAEGHVEVVKFLLEACKVNPFPKDRWNNTPMDEALHFGHHDVFKILQEYQVQYTPQGDSDDGKGNQTVHKNLDGLL

SEQ ID NO: 5 - protein, 8 aa, Artificial
  Conserved sequence
VLSPEAVR

SEQ ID NO: 6 - protein, 16 aa, Artificial
  C-terminus Unique to Mouse Glutaminase C
VSPESSDDTSTTVVYR

SEQ ID NO: 7 - protein, 26 aa, Artificial
  Isolated Peptide
GGTPPQQQQQQQQQPGASPPAAPGPK